(19) United States
(12) Patent Application Publication
(10) Pub. No.: US 2023/0305022 A1
(43) Pub. Date: Sep. 28, 2023

(54) Biomarkers in Primary Biliary Cholangitis

(71) Applicant: University of Newcastle upon Tyne, Tyne and Wear (GB)

(72) Inventors: David Jones, Tyne and Wear (GB); Steve Rushton, Tyne and Wear (GB)

(21) Appl. No.: 18/041,267

(22) PCT Filed: Aug. 10, 2021
(86) PCT No.: PCT/GB2021/052074

§ 371 Date: Feb. 10, 2023

(30) Foreign Application Priority Data
Aug. 11, 2020 (GB) 2012451.7

(51) Int. Cl. G01N 33/68; A61K 31/575

(52) U.S. Cl. CPC G01N 33/6863; A61K 31/575; G01N 2333/7155; G01N 2333/7158; G01N 2800/085; G01N 2800/52

(57) ABSTRACT

The present invention provides methods for predicting whether or not a test subject having primary biliary cholangitis (PBC) will benefit from advanced treatment in the form of a second-line agent used in addition to, or as an alternative to, ursodeoxycholic acid (UDCA). Associated methods for determining the therapeutic effect of a treatment regimen for PBC, methods for monitoring PBC progression, methods of PBC treatment, kits and assay devices are also provided.

The present invention provides methods for predicting whether or not a test subject having primary biliary cholangitis (PBC) will benefit from advanced treatment in the form of a second-line agent used in addition to, or as an alternative to, ursodeoxycholic acid (UDCA). Associated methods for determining the therapeutic effect of a treatment regimen for PBC, methods for monitoring PBC progression, methods of PBC treatment, kits and assay devices are also provided.

### BACKGROUND

Primary Biliary Cholangitis (PBC; previously known as primary biliary cirrhosis) is a chronic cholestatic liver disease with a significant inflammatory/immune component. The condition is characterised by damage to, and eventual destruction of, the small intra-hepatic bile ducts. Duct damage is accompanied by portal inflammation and in higher risk patients, interface hepatitis is also seen. There appear to be two elements to the disease process, and understanding the inter-relationship of these will be key moving forward in terms of disease therapy.

One component of the disease process is cholestatic injury, with damage to the biliary epithelial cells (BEC), mediated by hydrophobic bile acids, leading to duct injury and eventual ductopenia. BEC apoptosis and senescence can both be seen within affected liver (Tinmouth et al., 2002; Sasaki et al., 2010). The second component is the characteristic inflammatory response focused on the portal tracts, accompanied by the almost universal presence of autoantibody and autoreactive T-cell responses. The autoreactive B-cell and T-cell responses in PBC have been well characterised, with autoreactivity against the highly conserved mitochondrial self-antigen pyruvate dehydrogenase being almost universal in patients. The localisation of CD8+ T-cell responses directed at mitochondrial antigens around affected ducts, and the particular enrichment seen in early stages of the disease, supported by the strong evidence suggesting immune-genetic disease susceptibility (Cordell et al., 2015), have all pointed to a key role for the autoreactive immune response in PBC pathogenesis.

However, despite the strong evidence for a functional autoreactive immune response in PBC, immunotherapeutic approaches to treatment, using agents with a strong underpinning evidence basis from mechanistic and genetic studies, have proved disappointing in clinical trials (de Graaf et al., 2018). In contrast, anti-cholestatic therapy has been shown to be effective in a significant proportion of patients with ursodeoxycholic acid (UDCA) and Obeticholic Acid (OCA) bile-acid therapeutic agents approved and in widespread clinical use. At present, there has been little study of the inter-relationship of the two elements to PBC pathogenesis and, in particular, the impact of anti-cholestatic therapy on the inflammatory and immune processes in the disease.

The current treatment paradigm for treating PBC is initial treatment with 1^{st} line UDCA, which fully treats approximately 25% of PBC patients, and partially treats a further 35% of patients. Only once UDCA has failed are patients currently eligible for treatment with OCA, by which time there has been significant further damage to the liver, making cirrhosis complications and/or the need for a liver transplant more likely.

Prevention of PBC progression to advanced disease in the form of cirrhosis and/or ductopenia is a top priority. There is a clear clinical need for novel biomarkers that can be used to stratify PBC patients into UDCA responders and non-responders without the need to wait for clinical response status to become apparent thereby missing the opportunity to modify the disease course.

### BRIEF SUMMARY OF THE DISCLOSURE

The inventors have established a UK-PBC programme in which a large cohort of fully characterised PBC patients were recruited to study the mechanisms of disease as they relate to treatment, with a particular focus on why an important sub-group of patients do not respond to UDCA. In this two-stage study, the inventors set out to explore the serum inflammatory/immune proteome in PBC and its relationship to UDCA treatment response and non-response. There were 3 groups of participants: treatment naïve, newly presenting patients; patients established on UDCA therapy, stratified into UDCA responders and non-responders; and healthy age-matched controls. From this study, the inventors have identified the roles of key analytes in the different cohorts, differences between the cohorts and what this could mean for downstream therapies.

The inventors have identified that several senescence-associated secretory proteins are significantly elevated in the blood of PBC patients. They have also found that these proteins are only marginally improved by UDCA treatment, which fails to normalise BEC senescence in PBC patients. A number of these markers, namely, CCL20, CXCL11, CXCL10, CXCL9, CXCL13 CCL19, IL4RA, IL18R1, CD163, ACE2, CA5A, EpCAM, HAO1, DECR1, HAVCR1 (KIM1) and SCAMP3, also show significant differential expression in blood between PBC patients that are UDCA responders and those that are UDCA non-responders. One or more of these markers can therefore be used to stratify patients with PBC, and thus assist in selection of the optimal treatment regimen for these patients.

Although UDCA fails to normalise BEC senescence in PBC patients, the inventors show herein that OCA is directly anti-senescent. Advantageously, OCA may therefore be useful in reducing elevated levels of one or more of CCL20, CXCL11, CXCL10, CXCL9, CXCL13, CCL19, IL4RA, IL18R1, CD163, ACE2, CA5A, EpCAM, HAO1, DECR1, HAVCR1 (KIM1) or SCAMP3 in PBC patients. This is particularly relevant to PBC patients that are UDCA non-responders, which are shown herein to have elevated levels of these proteins. OCA treatment may be used to treat PBC in these patients, either alone, or in combination with UDCA.

In one aspect, the invention provides a method for predicting the level of response to ursodeoxycholic acid (UDCA) in a test subject having primary biliary cholangitis, the method comprising the steps of:
a) determining the level of one or more biomarker in a biological fluid sample from the test subject, wherein the one or more biomarker is selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3;
b) comparing the level of the one or more biomarker with a threshold level or range; and
c) predicting that:
   i) a test subject having a decreased level of the one or more biomarker compared to the threshold level or range is a UDCA responder; and
   ii) a test subject having an increased level of the one or more biomarker compared to the threshold level or range is a UDCA non-responder.

Suitably, the method may include the step of treating the subject for primary biliary cholangitis based on the prediction in step c).

Suitably, the method may comprise determining the level of at least two, three or four of the biomarkers.

Suitably, the at least two, three or four biomarkers may be selected from the group consisting of: CCL20, CXCL11, CXCL10 and CCL19.

Suitably, the at least two biomarkers may comprise CCL20 and CXCL11.

Suitably, the method may further comprise determining the level of CXCL9 in the biological fluid sample.

Suitably, for test subjects that are predicted to be UDCA non-responders, the method may further comprise selecting, or selecting and administering, an FXR agonist or a fibrate, optionally in combination with UDCA. Optionally, the FXR agonist may be obeticholic acid. Optionally, the fibrate may be a PPAR-alpha fibrate, a PPAR-beta fibrate, or a combination thereof. Examples of suitable fibrates are provided elsewhere herein and include bezafibrate, fenofibrate and gemfibrozil.

In one aspect, the invention also provides a method for determining the therapeutic effect of a treatment regimen for a test subject having primary biliary cholangitis, the method comprising:
a) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EpCAM, HAO1, DECR1, HAVCR1 and SCAMP3;
b) repeating step a) using a biological fluid sample obtained from the test subject after treatment for a time interval; and
c) comparing the level of biomarker determined in step a) to that determined in step b), and identifying that the treatment regimen has a therapeutic effect if there is no increase in the level of the one or more biomarker after treatment, or if there is a decrease in the level of the one or more biomarker after treatment.

Suitably, the method may include the step of treating the subject according to a prescribed treatment regimen for a time interval in between steps a) and b).

Suitably, the method may comprise determining the level of at least two or three biomarkers.

Suitably, the at least two or three biomarkers may be selected from the group consisting of: CCL20, CXCL11 and CXCL10.

Suitably, the at least two biomarkers may comprise CCL20 and CXCL11.

Suitably, the method may further comprise determining the level of CXCL9 and/or CCL19 in the biological fluid sample.

Suitably, the treatment regimen may comprise ursodeoxycholic acid (UDCA).

Suitably, a test subject having no increase in the level of the one or more biomarker after UDCA treatment, or having a decreased level of the one or more biomarker after UDCA treatment may be determined to be a UDCA responder. Suitably, a test subject having an increased level of the one or more biomarker after UDCA treatment may be determined to be a UDCA non-responder.

In one aspect, the invention also provides a method for monitoring the progression of primary biliary cholangitis in a test subject, the method comprising the steps of:
i) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EpCAM, HAO1, DECR1, HAVCR1 and SCAMP3;
ii) repeating step i) for the same test subject after a time interval; and
iii) comparing the biomarker levels identified in i) with the biomarker levels identified in ii), wherein a change in the biomarker levels from i) to ii) is indicative of a change in primary biliary cholangitis progression in the test subject.

Suitably, the method may include the step of treating the subject based on the comparison in step iii).

Suitably, the method may comprise determining the level of at least two or three biomarkers.

Suitably, the at least two or three biomarkers may be selected from the group consisting of: CCL20, CXCL11 and CXCL10.

Suitably, the at least two biomarkers may comprise CCL20 and CXCL11.

Suitably, the method may further comprise determining the level of CXCL9 and/or CCL19 in the biological fluid sample in each of steps i) and ii).

In one aspect, the use of one or more biomarkers selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3 is provided as a biological fluid biomarker for: predicting the level of response to ursodeoxycholic acid (UDCA) in a test subject having primary biliary cholangitis; or selecting a treatment regimen for a test subject having primary biliary cholangitis.

In one aspect, the invention also provides a method for treating a subject having primary biliary cholangitis, the method comprising: administering an FXR agonist or a fibrate to a subject, wherein the subject is identified as in need of treatment for primary biliary cholangitis based on having, in a biological fluid sample, an increased level of one or more biomarker selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3 compared to a threshold level or range, optionally wherein the FXR agonist is obeticholic acid, or the fibrate is bezafibrate, fenofibrate or gemfibrozil).

Suitably, the subject may be undergoing or has previously undergone treatment with ursodeoxycholic acid (UDCA).

In one aspect, the invention also provides a method for treating a subject having primary biliary cholangitis, the method comprising: administering to a subject ursodeoxycholic acid (UDCA), wherein the subject is identified as in need of treatment for primary biliary cholangitis based on having, in a biological fluid sample, a decreased level of one or more biomarker selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3 compared to a threshold level or range.

Suitably, in any aspect, the biological fluid sample may be a blood sample, optionally wherein the blood sample may be a serum or plasma sample.

Suitably, in any aspect, the level of biomarker may be determined at the protein level, optionally using a process selected from the group consisting of: ELISA assay, immunoblotting, lateral flow assay, protein microarray and mass spectrometry.

Suitably, in any aspect, the method or use may further comprise selecting, selecting and administering, altering, or terminating, a treatment regimen for the test subject based on the comparison of the level of the biomarker with the threshold level or range.

Suitably, in any aspect, for a test subject having an increased level of the one or more biomarker compared to the threshold level or range, the method or use may comprise selecting, or selecting and administering, a treatment regimen comprising an FXR agonist or a fibrate, optionally in combination with UDCA.

Suitably, in any aspect, the FXR agonist may be obeticholic acid.

Suitably, in any aspect, the fibrate may be selected from bezafibrate, fenofibrate or gemfibrozil.

Suitably, the level of one or more biomarker in a biological fluid sample from the subject may be determined in any of the methods described herein by contacting the biological fluid sample with an appropriate antibody specific to the biomarker of interest and detecting binding between the biomarker and the corresponding antibody (i.e. CCL20 binding to an anti-CCL20 antibody; CXCL11 binding to an anti-CXCL11 antibody; CXCL10 binding to an anti-CXCL10 antibody; CXCL13 binding to an anti-CXCL13 antibody; CCL19 binding to an anti-CCL19 antibody; CXCL9 binding to an anti-CXCL9 antibody; IL4RA binding to an anti-IL4RA antibody; IL18R1 binding to an anti-IL18R1 antibody; CD163 binding to an anti-CD163 antibody; ACE2 binding to an anti-ACE2 antibody; CA5A binding to an anti-CA5A antibody; EpCAM binding to an anti-EpCAM antibody; HAO1 binding to an anti-HAO1 antibody; DECR1 binding to an anti-DECR1 antibody; HAVCR1 binding to an anti-HAVCR1 antibody; or SCAMP3 binding to an anti-SCAMP3 antibody).

In one aspect, a kit is provided that is suitable for use in the methods or uses described herein, the kit comprising:
(i) a detectably labelled agent that specifically binds to CCL20 protein; and
(ii) one or more of:
   (a) a detectably labelled agent that specifically binds to CXCL11 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Suitably, the kit may further comprise one or more reagents for detecting the detectably labelled agent(s).

In one aspect, an assay device is provided that is suitable for use in the methods and uses described herein, the device comprising a surface with at least two detectably labelled agents located thereon, wherein the at least two detectably labelled agents are:
(i) a detectably labelled agent that specifically binds to CCL20 protein; and
(ii) one or more of:
   (a) a detectably labelled agent that specifically binds to CXCL11 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Suitably, the at least two detectably labeled agents may be located in separate zones on the surface.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows differential expression of serum proteins between UDCA-Treated PBC, Treatment Naïve PBC and Healthy Control Groups in a proteomics discovery study. (A) UDCA Tx (treated; n=416) vs HC (healthy controls; n=97), (B) Naïve Tx (n=68) vs HC and (C) UDCA Tx vs Naïve Tx. (D) Venn diagram summary of comparison between different cohorts. All analytes were thresholded for p<0.05 and 1.5 fold change and presented on a log scale. Red dots are significantly changed analytes >1.5 fold change (FC). P-values were adjusted for multiple comparisons according to the Benjamini Hochberg method.

FIG. 2 shows correlation of the PBC proteome with (A) alkaline phosphatase in UDCA-treated patients, (B) alkaline phosphatase in treatment-naïve patients and (C) UK-PBC 10-year mortality/transplant risk in UDCA-treated patients. Serum ALP significantly correlates with a high number of analytes in both UDCA-treated and treatment-naïve cohorts and UK-PBC risk score 10-year mortality risk correlates with analytes in the UDCA-treated group. P-values were adjusted for multiple comparisons on a log scale vs fold change of normalised protein expression (NPX) data.

FIG. 3 shows the UDCA-Treated PBC Proteome. Significant degrees of pathway enhancement are seen with particularly strong effects around chemokine function (Table 3b).

FIG. 4 shows up-regulated chemokines in the serum of UDCA non-responders in the discovery proteomics study. Response criterion used was Paris 1 (although the effect was the same for the example chemokine CCL20 regardless of the response criterion applied (FIG. 5)). All analytes were assayed via the Olink platform and generated normalised protein expression (NPX). P-values were calculated using an unpaired t-test with a Welch's correction. **** denotes p<0.0001, ** denotes p<0.001.

FIG. 5 shows differential CCL20 expression in subjects in the proteomics discovery study stratified for UDCA-response/non-response. The effect of increased chemokine level was seen irrespective of the response/non-response criteria used.

FIG. 6 shows a comparison of chemokine levels in UDCA responders and non-responders in a second, confirmatory study population. **** denotes p<0.0001.

FIG. 7 shows predictive value of serum chemokine levels for UDCA response status in the confirmatory study. All five chemokines identified in the discovery study as remaining elevated in PBC patients despite UDCA therapy, with significantly higher levels in UDCA non-responders than responders, were also highly predictive of UDCA response status in the confirmatory study using an independent study cohort.

FIG. 8 shows chemokine profiles in UDCA non-responders, UDCA responders and UDCA complete responders (i.e. those UDCA responders whose liver biochemistry had returned completely to normal). The majority of the UDCA non-responders showed significant elevation of all six of the studied chemokines (or of 5 of the 6). Numbers of elevated chemokines in individual UDCA responders was significantly lower, however over 50% of responders showed significant elevation of at least one chemokine. Significant elevation of at least one chemokine was still in seen in the group of UDCA complete responders.

FIG. 9 shows up-regulated proteins in serum of UDCA non-responders. (A-K) Analytes differentially regulated between Responders (R) and Non-responders (NR). Response was defined according to patient serum biochemistry, non-responders were classified as those patients post-Rx with an ALP measurement ×3 ULN. All analytes were assayed via the Olink platform and generated normalised protein expression (NPX). P-values were calculated using an unpaired t-test with a Welch's correction.

FIG. 10 shows that cholestasis in a murine model is associated with significant cellular senescence (p21 is a cellular senescence marker). Graph showing % p21+ hepatocytes in liver section from Sham, BDL, OCA and accompanying representative images. Scale bar equals 100 microns. P values were calculated using Anova with Tukey post-hoc t-test (D-F) and equal * P<0.05, ** P<0.01 or *** P<0.001. This is significantly reduced by OCA therapy. OCA is therefore anti-senescent. In contrast, bezafibrate is not anti-senescent, indeed in this model higher levels of senescence were seen than in the untreated cholestatic animals.

FIG. 11 shows that CXCL11 and CCL20 were strongly predictive of non-response status in a second validation cohort.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

PBC patients are currently all treated with UDCA as a first line therapeutic agent. However, a significant proportion of PBC patients do not respond positively to UDCA treatment and are thus classified as “UDCA non-responders”. For such patients, additional treatment with a second-line therapeutic agent such as an FXR agonist (e.g. OCA), or a fibrate (e.g. bezafibrate, fenofibrate or gemfibrozil), may be needed. In such patients, treatment with an FXR agonist (e.g. OCA) or a fibrate (e.g. bezafibrate, fenofibrate or gemfibrozil) may be in addition to, or as an alternative to, treatment with UDCA.

The inventors have surprisingly identified several new biomarkers that are elevated in the biological fluid (e.g. blood, such as serum or plasma) of PBC patients that are UDCA-non responders (compared to the levels of these biomarkers in PBC patients that are UDCA responders). The new biomarkers are CCL20, CXCL11, CXCL10, CXCL9, CXCL13, CCL19, IL4RA, IL18R1, CD163, ACE2, CA5A, EpCAM, HAO1, DECR1, HAVCR1 (KIM1) and SCAMP3.

One or more of these biomarkers can be used to predict the level of response to UDCA in a test subject having primary biliary cholangitis, and thus predict whether the test subject is a UDCA responder or a UDCA non-responder. One or more (e.g. two, three, four or five etc.) of these biomarkers can advantageously be used in any of the methods, kits, assays, or uses described herein.

Advantageously, these biomarkers may be used to predict the level of response to UDCA in test subject having PBC, where the test subject has already started UDCA treatment. For example, the biomarkers may be used to predict whether the ongoing UDCA treatment is effective. This may facilitate further treatment planning for the subject, such as deciding whether the UDCA treatment should be supplemented with an additional second line therapeutic agent (such as an FXR agonist (e.g. OCA) or a fibrate (e.g. bezafibrate, fenofibrate or gemfibrozil)), or whether the UDCA treatment should be replaced altogether (e.g. by a second line therapeutic agent such as an FXR agonist (e.g. OCA) or a fibrate (e.g. bezafibrate, fenofibrate or gemfibrozil)).

The biomarkers may alternatively be used to predict the level of response to UDCA in test subject having PBC, where the test subject is still treatment naïve for PBC (i.e. they have not yet started a treatment regimen for PBC). In such situations, the biomarkers may be used to predict whether UDCA treatment would be effective for the subject. In other words, the biomarkers can be used to facilitate treatment planning for the subject e.g. whether the subject should be treated with UDCA only, if UDCA treatment should be supplemented with an additional second line therapeutic agent such as an FXR agonist (e.g. OCA) or a fibrate (e.g. bezafibrate, fenofibrate or gemfibrozil), or if UDCA treatment should not be used (i.e. UDCA treatment should be replaced with a second line therapeutic agent such as an FXR agonist (e.g. OCA) or a fibrate (e.g. bezafibrate, fenofibrate or gemfibrozil)).

Methods for Predicting the Level of Response to UDCA in a Test Subject Having PBC

A method is provided herein for predicting the level of response to UDCA in a test subject having PBC.

The method comprises the steps of:
a) determining the level of one or more biomarker in a biological fluid sample from the test subject, wherein the one or more biomarker is selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3;
b) comparing the level of the one or more biomarker with a threshold level or range; and
c) predicting that:
   i) a test subject having a decreased level of the one or more biomarker compared to the threshold level or range is a UDCA responder; and
   ii) a test subject having an increased level of the one or more biomarker compared to the threshold level or range is a UDCA non-responder.

The term “subject” as used herein is preferably a mammal, such as a human.

The subject may be referred to herein as a patient. The terms “test subject”, “subject”, “individual”, and “patient” are used herein interchangeably. The subject can be symptomatic (e.g., the subject presents symptoms associated with primary biliary cholangitis), or the subject can be asymptomatic (e.g., the subject does not present symptoms associated with primary biliary cholangitis).

The terms “primary biliary cholangitis” or “PBC” refer to a chronic non-suppurative destructive granulomatous cholangitis with unknown etiology in which the pathology is more related to the medium-sized intrahepatic bile ducts rather than hepatocytes, resulting in cholestatic features of the disease with a high level of alkaline phosphatase (ALP) in serum (Kaplan et al., 2005). Inflammation usually starts adjacent the biliary system, resulting in cholestatic disease leading to fibrosis. Although other autoantibodies (e.g., ANA) may be detected in PBC, the anti-mitochondrial antibody (AMA) against acyltransferases of the inner mitochondrial membrane has high sensitivity and specificity for PBC (Kaplan et al. supra) and is reported in 95% of PBC cases. In addition, elevated immunoglobulins, especially IgM, as well as specific histologic features such as bile duct damage, ductopenia, and granulomatous portal inflammation are indicative of a PBC diagnosis. PBC is more common in women and is currently considered as a liver-specific autoimmune disease occurring in genetically predisposed individuals with association to other autoimmune conditions such as Sjogren syndrome and thyroid disease. PBC is generally not reported in children. “Children” as used herein refers to individuals under 12 years old.

The subject may be diagnosed with, be at risk of developing or present with symptoms of primary biliary cholangitis. The subject may have, or be suspected of having (e.g. present with symptoms or a history indicative or suggestive of), primary biliary cholangitis.

Typically, the subject has been diagnosed as having primary biliary cholangitis, using criteria known in the field. For example, the subject may have been diagnosed as having PBC due to elevation in serum alkaline phosphatase in the context of anti-mitochondrial antibody and/or characteristic bile duct lesions identified on liver biopsy. Appropriate methods for diagnosing PBC are well known in the art.

In particular examples, the subject has early stage primary biliary cholangitis. An example of an early stage of disease is when the subject has the initial symptoms of primary biliary cholangitis, including any symptoms indicative of a liver disease such as fatigue, right upper quadrant (RUQ) abdominal pain, nausea, pruritus, jaundice, and/or any abnormal levels of liver enzymes.

In other examples, the subject has advanced stages of PBC. An advanced stage signifying progression of the disease into a more severe form includes multiple stages where the initial stage does not have severe symptoms. PBC stages include: Portal Stage: Normal sized triads; portal inflammation, subtle bile duct damage. Granulomas—nodules filled with a variety of inflammatory cells—are often detected in this stage; Periportal Stage: Enlarged triads; periportal fibrosis and/or inflammation. Typically characterized by the finding of a proliferation of small bile ducts; Septal Stage. Active and/or passive fibrous septae; and Biliary Cirrhosis: Nodules present; garland or jigsaw pattern.

In one example, the subject is already undergoing therapy, e.g., undergoing therapy to treat suspected or diagnosed PBC, or undergoing therapy which places the subject at risk of developing PBC, an autoimmune disease and/or PBC. For example, the subject may already be undergoing a treatment regimen which includes administration of UDCA for treating PBC. In some examples, the subject may already be undergoing a treatment regimen which includes administration of UDCA, wherein it is suspected that the subject may not be fully benefiting from (e.g. not responsive to) UDCA treatment.

In other examples, the subject is PBC treatment naïve. In other words, the subject is not yet undergoing therapy to treat suspected or diagnosed PBC.

The methods are for predicting the level of response to UDCA in a test subject having primary biliary cholangitis.

The term “predicting” refers to the expected evolution of the treatment of a subject and refers to the prediction regarding the responsiveness of the subject to the treatment now or in the future (in order words, regarding the benefit of the treatment to the subject). It will be appreciated that a prediction might not always be accurate and/or informative. However, in the context of the invention, “predicting” requires that the prediction be accurate and/or informative regarding the responsiveness of the subject to the treatment in at least some of the a statistically significant part of subjects, for example in at least 30%, at least 40%, at least 50%, at least 60%, at least 70%, at least 80%, at least 90%, at least 95%, or more of the subjects. It will be appreciated that the prediction may be not solely based on the levels of the one or more biomarkers, and may be made in conjunction with, for example, other clinical observations, such as symptom severity, subject's age and family history. Suitably, the prediction may provide an accurate and/or informative prediction regarding the benefit of the treatment to the subject in a statistically significant proportion of the subjects. The amount that is statistically significant can be established by a person skilled in the art by using different statistical tools, for example, but not limited to, by determining confidence intervals, determining the significant p-value, Student's t-test or Fisher's discriminant function, non-parametric Mann-Whitney measurements, Spearman's correlation, logistic regression, linear regression, area under the ROC curve (AUC). Preferably, the confidence intervals are at least 90%, at least 95%, at least 97%, at least 98% or at least 99%. Preferably, the p-value is less than 0.1, than 0.05, than 0.01, than 0.005 or than 0.0001. In some examples, the prediction may be sufficiently accurate such that the term “predicting” can be used interchangeably with “determining” or “identifying”.

The methods are for predicting the level of response to UDCA in a test subject having PBC. The methods may be for predicting whether a subject that is currently undergoing UDCA treatment is responding to the treatment, and/or predicting whether they will respond to (ongoing) UDCA treatment in the future. Alternatively, the methods may be for predicting whether a PBC treatment naïve subject will respond to UDCA treatment in the future. Based on these predictions, a decision can be made on whether UDCA treatment should be started, continued, supplemented with additional treatments, or replaced, as appropriate.

As used herein “level of response” refers to the reaction of the subject to the treatment of interest. In the context of the invention, the level of response is generally in the context of UDCA treatment. Typically, therefore, the level of response is measured in the context of a treatment regimen which includes administration of UDCA. The methods can be used to predict that a test subject would benefit from treatment with UDCA (i.e. predict that they are a UDCA responder) or predict that a test subject would not benefit from treatment with UDCA (i.e. predict that they are a UDCA non-responder).

As used herein, “treatment regimen” refers to a structured treatment plan designed to improve and maintain the health of a subject. A treatment plan typically specifies the dosage of any medication that is to be administered to the subject, as well as specifying the schedule (e.g. frequency, time intervals between administration etc.) and duration of the treatment. A treatment regimen comprising UDCA therefore typically specifies the dose, schedule and duration of UDCA therapy that is to be administered to a patient. Appropriate treatment regimens comprising UDCA for treating PBC are well known in the art.

The term “responder” refers to a subject that shows a positive effect in response to a treatment. “UDCA responder” therefore refers to a subject that shows a positive effect in response to UDCA treatment. Responders and non-responders can be determined using pre-existing standard-of-care clinical and biochemical tests currently in use to determine whether a patient is responsive to treatment. For example, a PBC patient responsive to a treatment may have an alkaline phosphatase (ALP) blood/serum level under 200 units/liter. In another example, a PBC patient responsive to a treatment may be identified using the scoring system described in Carbone et. al., 2016. For the avoidance of doubt, a “UDCA responder” is defined by serum biochemical markers as outlined in standard clinical guidelines (for details see Hirschfield et al., 2018).

A “non-responder” refers to a subject that does not show a positive effect in response to a treatment. “UDCA non-responder” therefore refers to a subject that does not show a positive effect in response to UDCA treatment. A non-responder can be identified using the same criteria as for a “responder above”. In other words, a non-responder may have an alkaline phosphatase (ALP) blood/serum level above 200 units/liter. In certain cases, a threshold level of a biomarker can be determined by assaying PBC patients known to have responded to the treatment and PBC patients known to not have responded to the treatment as determined by analysis of liver biopsy sample from these patients.

Subjects that are UDCA non-responders are those whose PBC cannot be adequately treated with UDCA alone. In other words, they do not benefit from treatment with UDCA only. However, they may benefit from treatment with UDCA in combination with other second line PBC treatments, such as an FXR agonist (e.g. OCA) or a fibrate (e.g. bezafibrate, fenofibrate or gemfibrozil). Treatment regimens comprising UDCA and another PBC treatment, such as an FXR agonist (e.g. OCA) or a fibrate (e.g. bezafibrate, fenofibrate or gemfibrozil) may therefore be selected for such subjects. Alternatively, treatment regimens that do not comprise UDCA but do include another PBC treatment, such as an FXR agonist (e.g. OCA) or a fibrate (e.g. bezafibrate, fenofibrate or gemfibrozil) may also be selected for such subjects. In other words, UDCA may be replaced with another treatment such an FXR agonist (e.g. OCA) or a fibrate (e.g. bezafibrate, fenofibrate or gemfibrozil) for treating PBC in such subjects.

Subjects that are UDCA responders are those whose PBC can be effectively treated with UDCA alone. In other words, they do benefit from treatment with UDCA only. They do not require treatment with UDCA in combination with other second line PBC treatments, such as an FXR agonist (e.g. OCA) or a fibrate (e.g. bezafibrate, fenofibrate or gemfibrozil). Treatment regimens comprising UDCA as the only active ingredient may therefore be selected for such subjects.

Ursodeoxycholic acid (UDCA; C₂₄H₄₀O₄), also known as ursodiol, is a secondary bile acid, produced in humans and most other species from metabolism by intestinal bacteria. It is synthesized in the liver in some species and was first identified in bear bile. In purified form, it has been used to treat or prevent several diseases of the liver or bile ducts. UDCA can be chemically synthesized and is marketed under multiple trade names, including Ursetor®, Udikast®, Actibile®, Actigall®, Biliver®, Deursil®, Egyurso®, Udcasid®, Udiliv®, Udinorm®, Udoxyl®, Urso®, Urso Forte®, Ursocol®, Ursoliv®, Ursofalk®, Ursosan®, Ursoserinox®, Udimarin®, Ursonova®, and Stener®. It is typically administered by mouth.

In general, the methods described are in vitro methods that are performed using a sample that has already been obtained from the subject (i.e. the sample is provided for the method, and the steps taken to obtain the sample from the subject are not included as part of the method).

However, in some examples, the methods may include the step of providing a biological fluid sample from a subject.

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

The methods provided herein comprise providing a biological fluid sample (for example a blood sample, such as a serum or plasma sample) from a subject. The samples being tested in the methods described herein are also referred to as “test samples”.

As used herein, the terms “biological sample”, “test sample”, “sample” and variations thereof refer to a sample obtained or derived from a subject. For the purposes described herein, the sample is, or comprises, a biological fluid (also referred to herein as a bodily fluid) sample.

As used herein, the term “biological fluid sample” encompasses a blood sample. The term biological fluid sample also encompasses other bodily fluids such as a urine sample or a saliva sample.

A blood sample may be a whole blood sample, or a processed blood sample/a blood fraction e.g. serum, plasma etc. Methods for obtaining biological fluid samples (e.g. whole blood, serum, plasma, urine, saliva etc.) from a subject are well known in the art. For example, methods for obtaining blood samples from a subject are well known and include established techniques used in phlebotomy. The obtained blood samples may be further processed using standard techniques to obtain e.g. a serum sample, or a plasma sample. Advantageously, methods for obtaining biological fluid samples from a subject are typically low-invasive or non-invasive.

A whole blood sample is defined as a blood sample drawn from the human body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g. a change of up to 5%, up to 4%, up to 3%, up to 2%, up to 1% etc. Whole blood contains both the cell and fluid portions of blood. A whole blood sample may therefore also be defined as a blood sample with (substantially) all of its cellular components in plasma, wherein the cellular components (i.e. at least comprising the requisite white blood cells, red blood cells, platelets of blood) are intact.

Where the biological sample is a blood sample, the blood sample can be obtained from fresh blood or stored blood (e.g., in a blood bank). The biological sample can be a blood sample expressly obtained for the methods described herein or a blood sample obtained for another purpose which can be subsampled for the methods described herein. Cell free biological fluid samples include serum and plasma.

In a preferred example, the biological fluid sample is serum or plasma.

Samples can be manipulated after or during procurement, such as, by treatment with reagents (e.g., anti-coagulants), dilution, and/or enrichment for certain components for an analyte (s) to be assayed. Samples can be pre-treated as necessary by dilution in an appropriate buffer solution, concentrated if desired, or fractionated by any number of methods including but not limited to ultracentrifugation, fractionation by fast performance liquid chromatography (FPLC), or precipitation. Any of a number of standard aqueous buffer solutions, employing one of a variety of buffers, such as phosphate, Tris, or the like, at physiological pH can be used. In general, after isolation, samples (such as blood samples) are stored at −80° C. until assaying.

Methods for analysing (and optionally isolating, enriching for or extracting) protein biomarkers from blood, plasma, serum etc. samples have been described previously, see for example; Heitzer et al., 2019.

The methods provided herein include the step of determining the level of one or more biomarker in a biological fluid sample from the subject, wherein the one or more biomarker is selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3. The method may, in some examples further comprise determining the level of CXCL9 in the biological fluid sample as well.

A biomarker is an organic biomolecule (e.g. a protein, polypeptide, peptide, isomeric form thereof, immunologically detectable fragment thereof, corresponding nucleic acid molecule (e.g. mRNA, cDNA etc.)) which is differentially present in a sample taken from a subject having a disease as compared with a subject not having the disease. A biomarker is differentially present if the mean or median level of the biomarker in the different groups is calculated to be statistically significant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity.

Typically, the biomarker referred to herein is measured at the protein level. Details of each of the biomarkers is provided below.

Chemokine (C-C motif) ligand 20 (CCL20) is a small cytokine belonging to the CC chemokine family. It is also known as liver activation regulated chemokine (LARC), Macrophage Inflammatory Protein-3 (MIP3A), CKb4, Exodus, MIP-3-alpha, MIP-3a, SCYA20, ST38, and C-C motif chemokine ligand 20. It is strongly chemotactic for lymphocytes and weakly attracts neutrophils. CCL20 is implicated in the formation and function of mucosal lymphoid tissues via chemoattraction of lymphocytes and dendritic cells towards the epithelial cells surrounding these tissues. CCL20 elicits its effects on its target cells by binding and activating the chemokine receptor CCR6. Examples of human CCL20 include those comprising an amino acid sequence of UniProt Acc. No. P78556; and naturally-occurring variants thereof. CCL20 detection encompasses detection of full-length CCL20, as well as detection of naturally-occurring fragments or other metabolites of CCL20 found in a biological sample, and detection of fragments or other derivatives generated by manipulation of a biological sample, with the proviso that detection of such fragments, metabolites, or derivatives is specific for detection of CCL20. CCL20 fragments are usually at least 10, 15, 20, 25, 30, 35, 40, 45, 50, 55, or 60 amino acids or more in length. CCL20 may be detected by using an antibody that specifically binds to CCL20 in a body fluid sample of a human subject. Exemplary antibodies include commercially available anti-CCL20 antibody, such as a monoclonal or polyclonal anti-CCL20 antibody available from R&D Systems and other known antibody suppliers.

C-X-C motif chemokine 11 (CXCL11) is a protein that in humans is encoded by the CXCL11 gene. It is also known as H174, Interferon-inducible T-cell alpha chemoattractant (I-TAC), Interferon-gamma-inducible protein 9 (IP-9), IP9, SCYB11, SCYB9B and b-R1. It is a small cytokine belonging to the CXC chemokine family. It is highly expressed in peripheral blood leukocytes, pancreas and liver, with moderate levels in thymus, spleen and lung and low expression levels were in small intestine, placenta and prostate. Gene expression of CXCL11 is strongly induced by IFN-γ and IFN-β, and weakly induced by IFN-α. This chemokine elicits its effects on its target cells by interacting with the cell surface chemokine receptor CXCR3, with a higher affinity than do the other ligands for this receptor, CXCL9 and CXCL10. CXCL11 is chemotactic for activated T cells. Examples of human CXCL11 include those comprising an amino acid sequence of UniProt Acc. No. 014625; and naturally-occurring variants thereof. CXCL11 detection encompasses detection of full-length CXCL11, as well as detection of naturally-occurring fragments or other metabolites of CXCL11 found in a biological sample, and detection of fragments or other derivatives generated by manipulation of a biological sample, with the proviso that detection of such fragments, metabolites, or derivatives is specific for detection of CXCL11. CXCL11 fragments are usually at least 10, 15, 20, 25, 30, 35, 40, 45, 50, 55, or 60 amino acids or more in length. CXCL11 may be detected by using an antibody that specifically binds to CXCL11 in a body fluid sample of a human subject. Exemplary antibodies include commercially available anti-CXCL11 antibody, such as a monoclonal or polyclonal anti-CXCL11 antibody available from R&D Systems and other known antibody suppliers. C-X-C motif chemokine 10 (CXCL10) is an 8.7 kDa protein that in humans is encoded by the CXCL10 gene. It is also known as Interferon gamma-induced protein 10 (IP-10), small-inducible cytokine B10, C7, IFI10, INP10, SCYB10, crg-2, gIP-10, mob-1 and C-X-C motif chemokine ligand 10. CXCL10 is a small cytokine belonging to the CXC chemokine family. CXCL10 is secreted by several cell types in response to IFN-γ. These cell types include monocytes, endothelial cells and fibroblasts. CXCL10 has been attributed to several roles, such as chemoattraction for monocytes/macrophages, T cells, NK cells, and dendritic cells, promotion of T cell adhesion to endothelial cells, antitumor activity, and inhibition of bone marrow colony formation and angiogenesis. This chemokine elicits its effects by binding to the cell surface chemokine receptor CXCR3. Examples of human CXCL10 include those comprising an amino acid sequence of UniProt Acc. No. P02778; and naturally-occurring variants thereof. CXCL10 detection encompasses detection of full-length CXCL10, as well as detection of naturally-occurring fragments or other metabolites of CXCL10 found in a biological sample, and detection of fragments or other derivatives generated by manipulation of a biological sample, with the proviso that detection of such fragments, metabolites, or derivatives is specific for detection of CXCL10. CXCL10 fragments are usually at least 10, 15, 20, 25, 30, 35, 40, 45, 50, 55, or 60 amino acids or more in length. CXCL10 may be detected by using an antibody that specifically binds to CXCL10 in a body fluid sample of a human subject. Exemplary antibodies include commercially available anti-CXCL10 antibody, such as a monoclonal or polyclonal anti-CXCL10 antibody available from R&D Systems and other known antibody suppliers.

C-C motif chemokine ligand 19 (CCL19), also known as ELC, Macrophage inflammatory protein 3 beta (MIP3P), MIP-3P and SCYA19, is a ligand for C-C Motif Chemokine Receptor 7 (CCR7). The interaction between CCL19 and CCR7 is essential for the motility of mature DCs and T cells to the lymph nodes, establishment of a close physical contact between them, initiation of a primary immune response and finally proliferation of antigen-specific T cells. Examples of human CCL19 include those comprising an amino acid sequence of UniProt Acc. No. Q99731; and naturally-occurring variants thereof. CCL19 detection encompasses detection of full-length CCL19, as well as detection of naturally-occurring fragments or other metabolites of CCL19 found in a biological sample, and detection of fragments or other derivatives generated by manipulation of a biological sample, with the proviso that detection of such fragments, metabolites, or derivatives is specific for detection of CCL19. CCL19 fragments are usually at least 10, 15, 20, 25, 30, 35, 40, 45, 50, 55, or 60 amino acids or more in length. CCL19 may be detected by using an antibody that specifically binds to CCL19 in a body fluid sample of a human subject. Exemplary antibodies include commercially available anti-CCL19 antibody, such as a monoclonal or polyclonal anti-CCL19 antibody available from R&D Systems and other known antibody suppliers.

C-X-C motif chemokine 9 (CXCL9) is a cytokine that affects the growth, movement, or activation state of cells that participate in immune and inflammatory response. It is also known as Monokine Induced By Interferon-Gamma (MIG), CMK, Humig, SCYB9, crg-10, C-X-C motif chemokine ligand 9. CXCL9 is chemotactic for activated T-cells and binds to CXCR3. Examples of human CXCL9 include those comprising an amino acid sequence of Accession No. NP_002407.1 (UniProt Acc. No. Q07325-1); and naturally-occurring variants thereof. CXCL9 detection encompasses detection of full-length CXCL9, as well as detection of naturally-occurring fragments or other metabolites of CXCL9 found in a biological sample, and detection of fragments or other derivatives generated by manipulation of a biological sample, with the proviso that detection of such fragments, metabolites, or derivatives is specific for detection of CXCL9. CXCL9 fragments are usually at least 10, 15, 20, 25, 30, 35, 40, 45, 50, 55, or 60 amino acids or more in length. CXCL9 may be detected by using an antibody that specifically binds to CXCL9 in a body fluid sample of a human subject. Exemplary antibodies include commercially available anti-CXCL9 antibody, such as a monoclonal or polyclonal anti-CXCL9 antibody available from R&D Systems and other known antibody suppliers.

The interleukin 4 receptor (IL4RA) is a type I cytokine receptor that is also known as IL4R and CD124. IL4R is its human gene. This gene encodes the alpha chain of the interleukin-4 receptor, a type I transmembrane protein that can bind interleukin 4 and interleukin 13 to regulate IgE antibody production in B cells. Interactions of IL-4 with TNFα promote structural changes to vascular endothelial cells, thus playing an important role in tissue inflammation. Examples of human IL4RA include those comprising an amino acid sequence of UniProt Acc. No. P24394; and naturally-occurring variants thereof. IL4RA detection encompasses detection of full-length IL4RA, as well as detection of naturally-occurring fragments or other metabolites of IL4RA found in a biological sample, and detection of fragments or other derivatives generated by manipulation of a biological sample, with the proviso that detection of such fragments, metabolites, or derivatives is specific for detection of IL4RA. IL4RA fragments are usually at least 10, 15, 20, 25, 30, 35, 40, 45, 50, 55, or 60 amino acids or more in length. IL4RA may be detected by using an antibody that specifically binds to IL4RA in a body fluid sample of a human subject. Exemplary antibodies include commercially available anti-IL4RA antibody, such as a monoclonal or polyclonal anti-IL4RA antibody available from R&D Systems and other known antibody suppliers.

The interleukin-18 receptor 1 (IL-18R1) is an interleukin receptor of the immunoglobulin superfamily. IL18R1 is its human gene. IL18R1 is also known as CDw218a (cluster of differentiation w218a), CD218a, IL-1Rrp, IL18RA, IL1RRP, interleukin 18 receptor 1, IL-18R-alpha, IL18Ralpha2 and IL-18Ralpha. This receptor specifically binds interleukin 18 (IL18), and is essential for IL18 mediated signal transduction. IFN-alpha and IL12 are reported to induce the expression of this receptor in NK and T cells. Examples of human IL18R1 include those comprising an amino acid sequence of UniProt Acc. No. Q13478; and naturally-occurring variants thereof. IL18R1 detection encompasses detection of full-length IL18R1, as well as detection of naturally-occurring fragments or other metabolites of IL18R1 found in a biological sample, and detection of fragments or other derivatives generated by manipulation of a biological sample, with the proviso that detection of such fragments, metabolites, or derivatives is specific for detection of IL18R1. IL18R1 fragments are usually at least 10, 15, 20, 25, 30, 35, 40, 45, 50, 55, or 60 amino acids or more in length. IL18R1 may be detected by using an antibody that specifically binds to IL18R1 in a body fluid sample of a human subject. Exemplary antibodies include commercially available anti-IL18R1 antibody, such as a monoclonal or polyclonal anti-IL18R1 antibody available from R&D Systems and other known antibody suppliers.

CD163 (Cluster of Differentiation 163) is a protein that in humans is encoded by the CD163 gene. It is also known as M130, MM130 and SCARI1. Examples of human CD163 include those comprising an amino acid sequence of UniProt Acc. No. Q86VB7; and naturally-occurring variants thereof. CD163 detection encompasses detection of full-length CD163, as well as detection of naturally-occurring fragments or other metabolites of CD163 found in a biological sample, and detection of fragments or other derivatives generated by manipulation of a biological sample, with the proviso that detection of such fragments, metabolites, or derivatives is specific for detection of CD163. CD163 fragments are usually at least 10, 15, 20, 25, 30, 35, 40, 45, 50, 55, or 60 amino acids or more in length. CD163 may be detected by using an antibody that specifically binds to CD163 in a body fluid sample of a human subject. Exemplary antibodies include commercially available anti-CD163 antibody, such as a monoclonal or polyclonal anti-CD163 antibody available from R&D Systems and other known antibody suppliers.

Angiotensin-converting enzyme 2 (ACE2 or ACEH) is an enzyme attached to the cell membranes of cells in the lungs, arteries, heart, kidney, and intestines. The human version of the enzyme is often referred to as hACE2. Examples of human ACE2 include those comprising an amino acid sequence of UniProt Acc. No. Q9BYF1; and naturally-occurring variants thereof. ACE2 detection encompasses detection of full-length ACE2, as well as detection of naturally-occurring fragments or other metabolites of ACE2 found in a biological sample, and detection of fragments or other derivatives generated by manipulation of a biological sample, with the proviso that detection of such fragments, metabolites, or derivatives is specific for detection of ACE2. ACE2 fragments are usually at least 10, 15, 20, 25, 30, 35, 40, 45, 50, 55, or 60 amino acids or more in length. ACE2 may be detected by using an antibody that specifically binds to ACE2 in a body fluid sample of a human subject. Exemplary antibodies include commercially available anti-ACE2 antibody, such as a monoclonal or polyclonal anti-ACE2 antibody available from R&D Systems and other known antibody suppliers.

Carbonic anhydrase 5A (CA5A) is a protein that in humans is encoded by the CA5A gene. It is also known as CA5, CA5AD, CAV, CAVA, GS1-21A4.1 and carbonic anhydrase 5A. Carbonic anhydrases (CAs) are a family of zinc metalloenzymes that catalyze the reversible hydration of carbon dioxide. They participate in a variety of biological processes, including respiration, calcification, acid-base balance, bone resorption, and the formation of aqueous humor, cerebrospinal fluid, saliva, and gastric acid. They show extensive diversity in tissue distribution and in their subcellular localization. CA5A is localized in the mitochondria and expressed primarily in the liver. Examples of human CA5A include those comprising an amino acid sequence of UniProt Acc. No. P35218; and naturally-occurring variants thereof. CA5A detection encompasses detection of full-length CA5A, as well as detection of naturally-occurring fragments or other metabolites of CA5A found in a biological sample, and detection of fragments or other derivatives generated by manipulation of a biological sample, with the proviso that detection of such fragments, metabolites, or derivatives is specific for detection of CA5A. CA5A fragments are usually at least 10, 15, 20, 25, 30, 35, 40, 45, 50, 55, or 60 amino acids or more in length. CA5A may be detected by using an antibody that specifically binds to CA5A in a body fluid sample of a human subject. Exemplary antibodies include commercially available anti-CA5A antibody, such as a monoclonal or polyclonal anti-CA5A antibody available from R&D Systems and other known antibody suppliers.

Epithelial cell adhesion molecule (EpCAM) is a transmembrane glycoprotein mediating Ca2+-independent homotypic cell-cell adhesion in epithelia. It is also known as DIAR5, EGP-2, EGP314, EGP40, ESA, HNPCC8, KS1/4, KSA, M4S1, MIC18, MK-1, TACSTD1 and TROP1. EpCAM is also involved in cell signaling, migration, proliferation, and differentiation. Examples of human EpCAM include those comprising an amino acid sequence of UniProt Acc. No. P16422; and naturally-occurring variants thereof. EpCAM detection encompasses detection of full-length EpCAM, as well as detection of naturally-occurring fragments or other metabolites of EpCAM found in a biological sample, and detection of fragments or other derivatives generated by manipulation of a biological sample, with the proviso that detection of such fragments, metabolites, or derivatives is specific for detection of EpCAM. EpCAM fragments are usually at least 10, 15, 20, 25, 30, 35, 40, 45, 50, 55, or 60 amino acids or more in length. EpCAM may be detected by using an antibody that specifically binds to EpCAM in a body fluid sample of a human subject. Exemplary antibodies include commercially available anti-EpCAM antibody, such as a monoclonal or polyclonal anti-EpCAM antibody available from R&D Systems and other known antibody suppliers.

Hydroxyacid oxidase (glycolate oxidase) 1 (HAO1) is a protein that in humans is encoded by the HAO1 gene. It is also known as HAOX1, GOX, GOX1, Hydroxyacid oxidase (glycolate oxidase) 1 and hydroxyacid oxidase 1. The HAO1 gene is expressed primarily in liver and pancreas and the encoded protein is most active on glycolate, a two-carbon substrate. Glycolate oxidase oxidizes glycolic acid to glyoxylate, and can also oxidize glyoxylate into oxalate. Examples of human HAO1 include those comprising an amino acid sequence of UniProt Acc. No. Q9UJM8; and naturally-occurring variants thereof. HAO1 detection encompasses detection of full-length HAO1, as well as detection of naturally-occurring fragments or other metabolites of HAO1 found in a biological sample, and detection of fragments or other derivatives generated by manipulation of a biological sample, with the proviso that detection of such fragments, metabolites, or derivatives is specific for detection of HAO1. HAO1 fragments are usually at least 10, 15, 20, 25, 30, 35, 40, 45, 50, 55, or 60 amino acids or more in length. HAO1 may be detected by using an antibody that specifically binds to HAO1 in a body fluid sample of a human subject. Exemplary antibodies include commercially available anti-HAO1 antibody, such as a monoclonal or polyclonal anti-HAO1 antibody available from R&D Systems and other known antibody suppliers.

2,4 Dienoyl-CoA reductase (also known as DECR1) is an enzyme which in humans is encoded by the DECR1 gene. DECR1 participates in the beta oxidation and metabolism of polyunsaturated fatty enoyl-CoA esters. Examples of human DECR1 include those comprising an amino acid sequence of UniProt Acc. No. Q16698; and naturally-occurring variants thereof. DECR1 detection encompasses detection of full-length DECR1, as well as detection of naturally-occurring fragments or other metabolites of DECR1 found in a biological sample, and detection of fragments or other derivatives generated by manipulation of a biological sample, with the proviso that detection of such fragments, metabolites, or derivatives is specific for detection of DECR1. DECR1 fragments are usually at least 10, 15, 20, 25, 30, 35, 40, 45, 50, 55, or 60 amino acids or more in length. DECR1 may be detected by using an antibody that specifically binds to DECR1 in a body fluid sample of a human subject. Exemplary antibodies include commercially available anti-DECR1 antibody, such as a monoclonal or polyclonal anti-DECR1 antibody available from R&D Systems and other known antibody suppliers. Hepatitis A virus cellular receptor 1 (HAVCR-1) also known as T-cell immunoglobulin and mucin domain 1 (TIM-1) is a protein that in humans is encoded by the HAVCR1 gene. It is also known as KIM-1 (Kidney Injury Molecule-1) and is a protein the most highly upregulated in injured kidneys by various types of insults. Examples of human KIM-1 include those comprising an amino acid sequence of UniProt Acc. No. Q96D42; and naturally-occurring variants thereof. KIM-1 detection encompasses detection of full-length KIM-1, as well as detection of naturally-occurring fragments or other metabolites of KIM-1 found in a biological sample, and detection of fragments or other derivatives generated by manipulation of a biological sample, with the proviso that detection of such fragments, metabolites, or derivatives is specific for detection of KIM-1. KIM-1 fragments are usually at least 10, 15, 20, 25, 30, 35, 40, 45, 50, 55, or 60 amino acids or more in length. KIM-1 may be detected by using an antibody that specifically binds to KIM-1 in a body fluid sample of a human subject. Exemplary antibodies include commercially available anti-KIM-1 antibody, such as a monoclonal or polyclonal anti-KIM-1 antibody available from R&D Systems and other known antibody suppliers.

Secretory carrier-associated membrane protein 3 (SCAMP3 or C1orf3) is a protein that in humans is encoded by the SCAMP3 gene. This gene product belongs to the SCAMP family of proteins which are secretory carrier membrane proteins. They function as carriers to the cell surface in post-golgi recycling pathways. Different family members are highly related products of distinct genes, and are usually expressed together. These findings suggest that the SCAMPs may function at the same site during vesicular transport rather than in separate pathways. Two transcript variants encoding different isoforms have been found for this gene. SCAMP3 has been shown to interact with NEDD4. Examples of human SCAMP3 include those comprising an amino acid sequence of UniProt Acc. No. 014828; and naturally-occurring variants thereof. SCAMP3 detection encompasses detection of full-length SCAMP3, as well as detection of naturally-occurring fragments or other metabolites of SCAMP3 found in a biological sample, and detection of fragments or other derivatives generated by manipulation of a biological sample, with the proviso that detection of such fragments, metabolites, or derivatives is specific for detection of SCAMP3. SCAMP3 fragments are usually at least 10, 15, 20, 25, 30, 35, 40, 45, 50, 55, or 60 amino acids or more in length. SCAMP3 may be detected by using an antibody that specifically binds to SCAMP3 in a body fluid sample of a human subject. Exemplary antibodies include commercially available anti-SCAMP3 antibody, such as a monoclonal or polyclonal anti-SCAMP3 antibody available from R&D Systems and other known antibody suppliers.

Chemokine (C-X-C motif) ligand 13 (CXCL13) is a protein ligand that in humans is encoded by the CXCL13 gene. It is also known as also known as B lymphocyte chemoattractant (BLC) or B cell-attracting chemokine 1 (BCA-1), ANGIE, ANGIE2, BLR1L and SCYB13. CXCL13 is a small chemokine belonging to the CXC chemokine family. As its name suggests, this chemokine is selectively chemotactic for B cells belonging to both the B-1 and B-2 subsets, and elicits its effects by interacting with chemokine receptor CXCR5. Examples of human CXCL13 include those comprising an amino acid sequence of UniProt Acc. No. 043927; and naturally-occurring variants thereof. CXCL13 detection encompasses detection of full-length CXCL13, as well as detection of naturally-occurring fragments or other metabolites of CXCL13 found in a biological sample, and detection of fragments or other derivatives generated by manipulation of a biological sample, with the proviso that detection of such fragments, metabolites, or derivatives is specific for detection of CXCL13. CXCL13 fragments are usually at least 10, 15, 20, 25, 30, 35, 40, 45, 50, 55, or 60 amino acids or more in length. CXCL13 may be detected by using an antibody that specifically binds to CXCL13 in a body fluid sample of a human subject. Exemplary antibodies include commercially available anti-CXCL13 antibody, such as a monoclonal or polyclonal anti-CXCL13 antibody available from R&D Systems and other known antibody suppliers.

The methods described herein include the step of determining the level of the one or more biomarker in a biological fluid sample. Conventional “determining” methods may include sending a clinical sample(s) to a commercial laboratory for measurement of the biomarker levels in the biological fluid sample, or the use of commercially available assay kits for measuring the biomarker levels in the biological fluid sample. Exemplary kits and suppliers will be apparent to a person of skill in the art. In various examples, biomarkers may be determined, detected and/or quantified using ELISA assays or lateral flow devices, such as for point-of-care use, as well as spot check colorimetric tests.

The level of biomarker present in the biological fluid sample may be determined by e.g. assaying the amount of protein biomarker present in the sample. Assays for measuring the amount of a specified protein are well known in the art and include direct or indirect measures.

The level of protein biomarker in a sample may also be determined by determining the level of protein biomarker activity in a sample. Accordingly, protein “level” encompasses both the amount of protein per se, or its level of activity.

By way of example, the level of a protein biomarker in a biological fluid sample can be determined (e.g., measured) by any suitable methods and materials known in the art, including, for example, a process selected from the group consisting of mass spectrometry, immunoassays, enzymatic assays, spectrophotometry, colorimetry, fluorometry, bacterial assays, protein microarrays, compound separation techniques, or other known techniques for determining the presence and/or quantity of an analyte. Examples of relevant techniques include enzyme linked immunosorbent assays (ELISAs), immunoprecipitation, immunofluorescence, enzyme immunoassay (EIA), radioimmunoassay (RIA), Western blot analysis, and Lateral Flow (using e.g. Lateral Flow Devices (LFDs) utilizing a membrane bound antibody specific to the protein biomarker). Preferably, the level of a protein biomarker in a biological fluid sample is measured by ELISA or lateral flow.

In an example, the methods described herein determine the level of two or three or four of the specified biomarkers.

In one example, the at least two, three or four biomarkers may be selected from the group consisting of: CCL20, CXCL11, CXCL10 and CCL19. For example, the at least two biomarkers may comprise CCL20 and CXCL11. In addition, the level of CXCL9 in the biological fluid sample may also be determined.

For example, the method may determine the level of CCL20 and CXCL11; CCL20 and CXCL10; CCL20 and CCL19; or CCL20 and CXCL9.

The method may determine the level of CCL20, CXCL11 and CXCL10. Alternatively, the method may determine the level of CCL20, CXCL11 and CCL19. Alternatively, the method may determine the level of CCL20, CXCL11 and CXCL9.

The method may determine the level of CCL20, CXCL11, CXCL10 and CCL19. Alternatively, the method may determine the level of CCL20, CXCL11, CCL19 and CXCL9. Alternatively, the method may determine the level of CCL20, CXCL11, CXCL10 and CXCL9.

The method may determine the level of CCL20, CXCL11, CXCL10, CCL19 and CXCL9.

In a further example, the method may determine the level of CXCL11 and CXCL10; CXCL11 and CCL19; or CXCL11 and CXCL9.

In a further example, the method may determine the level of CXCL10 and CCL19; or CXCL10 and CXCL9.

In a further example, the method may determine the level of CCL19 and CXCL9.

In any of the above examples, the method may determine CXCL13.

The methods described herein comprise the step of comparing the level of the at least one biomarker (i.e. its amount per se or its activity) in the biological fluid sample (test sample) with a threshold level (or range) for the same biomarker.

As used herein, “biomarker threshold level”, “threshold level”, “biomarker control level” or “control level” may also be referred to as a “cutoff value”, “control value” or “threshold value”. It refers to a biomarker level that can be used to distinguish between a first “condition” and a second “condition” (e.g., wherein the first condition may be individuals who are UDCA responders and the second condition may be individuals who are UDCA non-responders) such that a biomarker level in a sample that is above the threshold level indicates an increased likelihood of the individual having the second condition (e.g. being a UDCA non-responder and thus requiring additional or alternative treatment for PBC than that provided by UDCA alone). Thus, a “threshold level” refers to an assay value (e.g., level of a biomarker), which is an approximate value that distinguishes the likelihood that a condition is present in the individual tested from the likelihood that a condition is not present in the individual tested, with a preselected specificity and/or sensitivity. Notably, the threshold may be a single value (“threshold level”) or a range of values (“threshold range”).

For example, a biomarker threshold level (or range) can represent an approximate level (or range) of a biomarker that detects affected subjects at a desired sensitivity (e.g., at least 55%, at least about 60%, at least 70%, or at least 80% or more). Thus, for example, an individual having a biomarker level that is greater than a threshold level (or range) has at least about 60% or greater likelihood of having that condition. It will be appreciated that the precise number value for threshold values (or ranges) can vary with the type of assay and reagents used to detect the biomarkers as well as the sensitivity and specificity desired from the assay. However, regardless of the assay and reagents used, the correlations between a threshold level (or range) and likelihood of a disease state (e.g., a PBC patient that is a UDCA non-responder or UDCA responder) will be present regardless of the assays and reagents used. Thus, as long as the test samples are assayed for the biomarker of interest using an assay platform and reagents of the same general type (e.g., protein assay) and similar sensitivity as the assay platform and reagents used to determine the threshold level (or range) of the biomarker, the findings upon which the methods described herein are based will be preserved. Appropriate threshold levels (or threshold ranges) can be determined using routine methods known in the art, e.g., by assaying levels of the biomarker(s) of interest in control populations, in PBC subjects known to be UDCA responders, and in PBC subjects known to be UDCA non-responders. Through application of statistical analysis, such methods can be used to identify a biomarker level that is present in at least 20%, at least 25%, at least 30%, at least 40%, at least 50%, at least 55%, at least 60%, at least 70%, at least 80%, or at least 90% or more of PBC patients that do not respond to UDCA therapy and which provides a specificity of at least 80%, at least 90%, at least 95%, at least 98%, at least 99%, or at least 100%.

Typically, the threshold level (or range) of the biomarker of interest may be determined by assaying the level of the biomarker in PBC patients that are known to respond to UDCA therapy and assaying the level of the biomarker in PBC patients that are known to be non-responders to UDCA therapy. The levels of the biomarker in such patients can be used to identify the range of biomarker levels that are observed in non-responders, which can be used to set the threshold level (or range).

The individuals used to generate the threshold level (or range) are typically of the same species, age or sex as the subject from which the test sample is obtained.

A threshold level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of a biomarker of a population of individuals comprising the subject to be investigated. How to calculate a suitable reference value, preferably, the average or median, is well known in the art. The population of subjects referred to before shall comprise a plurality of individuals, preferably, at least 5, 10, 50, 100, 1,000 subjects.

Suitably, the level of the specific biomarker detected in a sample (e.g. a test sample, a control sample etc.) may be normalized by adjusting the measured level (amount or activity) of the biomarker using the level of a reference protein in the same sample, wherein the reference protein is not a marker itself (it is e.g., a protein that is constitutively expressed). This normalization allows the comparison of the biomarker level in one sample to another sample, or between samples from different sources. This normalized level can then optionally be compared to the threshold level or range.

For example, when measuring a protein biomarker in a whole blood sample the biomarker may be expressed as an absolute concentration or, alternatively, it may be normalized against a known protein constitutively expressed in whole blood such as albumin, immunoglobulins or plasma protein concentration.

As another example, when measuring a protein biomarker in a serum sample the biomarker may be expressed as an absolute concentration or, alternatively, it may be normalized against a known protein constitutively expressed in serum.

The methods described herein include the step of predicting that the test subject is a UDCA responder if the test subject has a decreased level of the one or more biomarker compared to the threshold level or range. Conversely, if the test subject has an increased level of the one or more biomarker compared to the threshold level or range, the prediction is that the test subject is a UDCA non-responder (and thus requires additional or alternative treatment for PBC than that provided by UDCA alone).

The terms “decrease”, “decreased” “reduced”, “reduction” or “down-regulated”, “lower”, “less than” are all used herein generally to mean a decrease by a statistically significant amount. However, for avoidance of doubt, “reduced”, “reduction”, “decreased” or “decrease” etc. means a decrease by at least 10% as compared to a reference level/control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a threshold value or range), or any decrease between 10-100% as compared to a threshold value or range, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a threshold value or range.

The terms “increased”, “increase” or “up-regulated”, “higher” etc. are all used herein to generally mean an increase by a statically significant amount; for the avoidance of any doubt, the terms “increased” or “increase” means an increase of at least 10% as compared to a threshold value or range, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a threshold value or range, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a threshold value or range.

The methods can further comprise selecting, and optionally administering, a treatment regimen for the subject based on the comparison of the levels of the biomarkers with the threshold level (or range). Treatment can include, for example, administration of UDCA, either on its own, or with additional PBC therapeutic agents, such as an appropriate FXR agonist, such as obeticholic acid (OCA), or a different PBC therapeutic agent such as a fibrate (e.g. bezafibrate, fenofibrate or gemfibrozil). The treatment can therefore include a combination of UDCA and other PBC therapeutic agents. In other cases, the treatment may include PBC therapeutic agents other than UDCA (in other words, an FXR agonist such as OCA, or a different PBC therapeutic agent such as a fibrate (e.g. bezafibrate, fenofibrate or gemfibrozil), without the addition of UDCA).

As an example, for test subjects that are predicted to be UDCA non-responders, the method may further comprise selecting, or selecting and administering, an FXR agonist (e.g. OCA) or a fibrate (e.g. bezafibrate, fenofibrate or gemfibrozil). The FXR agonist (e.g. OCA) or fibrate (e.g. bezafibrate, fenofibrate or gemfibrozil) may be selected, or selected and administered, in combination with UDCA, or without UDCA.

An FXR agonist is a compound that activates the farnesoid X receptor (FXR). Typically, FXR agonists act directly on and activate the FXR, for example, the FXR agonist may be a compound such as a small molecule that acts directly on, e.g. binds, and activates the FXR. Methods for identifying appropriate FXR agonists are well known in the art, and include assaying for FXR activity in the presence and absence of the potential agonist and determining whether there is an increase in FXR activity. FXR is a nuclear receptor expressed in the liver, intestine, kidney, and adipose tissue that regulates a wide variety of target genes critically involved in the control of bile acid synthesis and transport, lipid metabolism, and glucose homeostasis. In the context of treating PBC, FXR activation suppresses de novo synthesis of bile acids in the hepatocytes as well as increases the transport of bile acids out of hepatocytes, thereby reducing exposure of the hepatocytes to bile acid. Suitable FXR agonists for treatment of PBC include obeticholic acid. Obeticholic acid (abbreviated to OCA, trade name Ocaliva®), is a semi-synthetic bile acid analogue which has the chemical structure 6α-ethyl-chenodeoxycholic acid (C₂₆H₄₄O₄). In clinical trials, OCA has proven to be effective as a second line therapy in patients with an unsatisfactory response to UDCA, termed “non-responders” (POISE study; NCT01473524) and is now approved for use as a second line therapy in both Europe and America. Other suitable FXR agonists are known in the field, including Tropifexor® (Novartis), Cilofexor® (Gilead) and EDP-305® (Enanta).

Fibrates (derivatives of fibrinic acid) are lipid regulating agents which constitute a class of amphipathic carboxylic acids. They are regarded as prodrugs and are metabolized in vivo to their active metabolites. Fibrates are commonly used for a range of metabolic disorders, mainly hypercholesterolemia (high cholesterol), and are therefore hypolipidemic agents. Fibrates are used in accessory therapy in many forms of hypercholesterolemia, usually in combination with statins.

The clinical effects of fibrates are caused by changes in the transcription of genes that play important roles in lipid metabolism. Fibrates activate peroxisome proliferator-activated receptors (“PPAR”), especially PPARα. PPARα is also referred to as PPAR-alpha and PPARα. The PPARs are a class of intracellular receptors that modulate carbohydrate and fat metabolism and adipose tissue differentiation. The PPARs belong to the family of nuclear hormone receptors. Activating PPARs induces the transcription of a number of genes that facilitate lipid metabolism. Fibrates are structurally and pharmacologically related to the thiazolidinediones, a novel class of anti-diabetic drugs that also act on PPARs (more specifically PPARγ).

As used herein, the term “fibrate” refers to a class of therapeutic compounds that include, without limitation, therapeutic compounds that are cholesterol-lowering drugs that are primarily effective in lowering triglycerides and, to a lesser extent, in increasing HDL-cholesterol levels. The term “fibrates” encompasses fibric acid derivatives (e.g. fenofibric acid or dofibric acid) and pharmaceutically acceptable salts and esters of such fibric acid derivatives.

Any suitable fibrate may be used in accordance with the present invention. Methods for identifying suitable fibrates are well known in the art. Examples of suitable fibrates include PPAR-alpha agonists, such as bezafibrate and fenofibrate, and PPAR-delta agonists, such as gemfibrozil and seladelpar.

“PPAR-alpha agonist” refers to a compound or composition which activates PPAR-alpha. Typically, when combined with PPAR-alpha, PPAR-alpha agonists directly or indirectly (preferably binding directly to PPAR-alpha) stimulate or increase an in vivo or in vitro reaction typical for the receptor, e.g. transcriptional regulation activity. “PPAR-alpha agonist” may be used interchangeably with “PPARa agonist” and “PPARα agonist”.

PPAR-alpha agonists include fibrate compounds such as, but not limited to fenofibrate, bezafibrate, clofibrate, ciprofibrate, and analogues, derivatives and pharmaceutically acceptable salts thereof.

Bezafibrate (C₁₉H₂₀ClNO₄) is a hypolipidemic fibrate drug that is approved for PBC treatment and has been shown to be beneficial when used in combination with UDCA. It is therefore a well-known second line option for PBC. It is marketed as Bezalip® as well as other brand names. Analogues, derivatives and pharmaceutically acceptable salts of bezafibrate are also encompassed.

Fenofibrate (C₂₀H₂₁ClO₄) is indicated as adjunct therapy to diet for the treatment of patients with primary hypercholesterolemia or mixed dyslipidemia. The effects of fenofibrate observed in clinical practice have been explained in vivo in transgenic mice and in vitro in human hepatocyte cultures by the activation of peroxisome proliferator activated receptor a (PPARα). Through this mechanism, fenofibrate increases lipolysis and elimination of triglyceride-rich particles from plasma by activating lipoprotein lipase and reducing production of apolipoprotein CIII (an inhibitor of lipoprotein lipase activity). The term “fenofibrate” is used to denote both the fenofibric acid and the salified or esterified form of this compound or analogues and derivatives thereof, Fenofibrate is commercially available in Europe (Lipanthyl®) 1975 and in the USA (TriCor®).

“PPAR-delta agonist” refers to a compound or composition which activates PPAR-delta (PPAR-delta is used interchangeably with PPAR-δ, additionally PPAR-β or PPAR-beta may also be used to refer to PPAR-delta in the art). Typically, PPAR-delta agonists directly or indirectly (preferably by binding directly to PPAR-delta) stimulates or increases an in vivo or in vitro reaction typical for the receptor, e.g. transcriptional regulation activity, as measured by an assay known to one skilled in the art. For example, a PPAR-delta agonist may induce a level of gene transcription comparable to endogenous ligands such as retinoic acid or comparable to a standard reference PPAR-delta agonist such as carbacyclin. “PPAR-delta agonist” may be used interchangeably with “PPAR-δ agonist”.

PPAR-delta agonists include fibrate compounds such as, but not limited to gemfibrozil, seladelpar, and analogues, derivatives and pharmaceutically acceptable salts thereof.

Gemfibrozil (C₁₅H₂₂O₃) is an FDA-approved lipid-lowering drug, is known to reduce the level of triglycerides in the blood circulation and decrease the risk of hyperlipidemia (Robins et al. 2001, Rubins & Robins 1992, Rubins et al. 1999). Gemfibrozil is marketed as Lopid® as well as other brand names. Analogues, derivatives and pharmaceutically acceptable salts of gemfibrozil are also encompassed.

Seladelpar ((R)-2-(4-((2-ethoxy-3-(4-(trifluoromethyl)phenoxy)propyl)-sulfanyl)-2-methylphenoxy)acetic acid) is an orally active, potent (2 nM) agonist of PPAR-delta; and is specific, being >600-fold and >2500-fold more potent at the PPAR-delta receptor than at the PPAR-alpha and PPAR-gamma receptors. Seladelpar is also known as MBX-8025 and is being investigated for use in several applications, including PBC. Analogues, derivatives and pharmaceutically acceptable salts of seladelpar are also encompassed.

In an alternative example, where the method predicts that the test subject is a UDCA responder, the method may further comprise selecting, or selecting and administering, a treatment regimen comprising UDCA to the subject.

In one example, the methods described herein may comprise:
a) selecting;
b) selecting and administering;
c) altering; or
d) terminating
a treatment regimen for the test subject based on the comparison of the level of the biomarker(s) in the test subject with the threshold level or range.

In other words, the level of biomarker(s) observed in a sample from a patient can be useful when making decisions on what treatment should be selected for that patient. The methods can therefore include the step of selecting an appropriate treatment regimen for the patient (based on their biomarker levels), and optionally may include the step of administering the selected treatment to the patient. In some cases, the biomarker levels will indicate the patient's current treatment regimen is not optimal and therefore should be altered (e.g. doses or frequency of treatment increased/decreased, or addition of a supplementary medication etc), or terminated (e.g. if the current treatment regimen is not beneficial and should be stopped).

For example, where the method determines that the test subject has an increased level of the one or more biomarker compared to the threshold level or range, the method may comprise selecting, or selecting and administering, a treatment regimen comprising an FXR agonist (e.g. OCA) or another PBC therapeutic agent such as a fibrate (e.g. bezafibrate, fenofibrate or gemfibrozil), either alone or more typically in combination with UDCA (e.g. FXR agonist plus UDCA; fibrate plus UDCA). In this example, the subject may be PBC treatment naïve (and the method therefore includes selecting, or selecting and administering, their first PBC treatment regimen).

Alternatively, the subject may already be undergoing or have previously undergone treatment for PBC (and the method therefore selects, or selects and administers, their new PBC treatment regimen, where the selection of a new PBC treatment may comprise altering and/or terminating their previous treatment regimen). For example, the subject may already be undergoing or have previously undergone UDCA treatment for PBC. In this example, where the method determines that the test subject has an increased level of the one or more biomarker compared to the threshold level or range, the method may comprise altering the treatment, wherein altering the treatment comprises supplementing the UDCA treatment with a second line therapeutic agent (such as an FXR agonist (e.g. OCA) or a fibrate (e.g. bezafibrate, fenofibrate or gemfibrozil)), or terminating the UDCA treatment and replacing it with a second line therapeutic agent (such as an FXR agonist (e.g. OCA) or a fibrate (e.g. bezafibrate, fenofibrate or gemfibrozil)).

As used herein, the terms “treat”, “treating” and “treatment” are taken to include an intervention performed with the intention of preventing the development or altering the pathology of a condition, disorder or symptom (i.e. in this case PBC). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the symptoms of PBC, for example of at least 5%, 10%, 20%, 30%, 40%, 50%, 60%, 70%, 80%, 90% or 100% when compared to the symptoms before treatment. In the context of PBC, appropriate treatment may include administration of one or more of 1) UDCA, 2) an appropriate FXR agonist (such as obeticholic acid (OCA)), or 3) an alternative PBC therapeutic agent such as a fibrate (e.g. bezafibrate, fenofibrate or gemfibrozil). Accordingly, the method may include the step of administering one or more of these treatments to the subject. Other suitable treatments are well known to a person of skill in the art and depend on the specific symptoms of the subject.

When a therapeutic agent or other treatment is administered, it is administered in an amount and/or for a duration that is effective to treat PBC or to reduce the likelihood (or risk) of PBC developing in the future. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes (e.g., biomarkers as described herein). It should be understood that the therapeutic agents described herein are used to treat and/or prevent PBC. Thus, in some cases, they may be used prophylactically in subjects at risk of developing PBC. Thus, in some cases, an effective amount is that amount which can lower the risk of, slow or perhaps prevent altogether the development of PBC. It will be recognized when the therapeutic agent is used in acute circumstances, it is used to prevent one or more medically undesirable results that typically flow from such adverse events. Methods for selecting a suitable treatment, an appropriate dose thereof and modes of administration will be apparent to one of ordinary skill in the art.

The medications or treatments described herein can be administered to the subject by any conventional route, including by mouth, injection or by gradual infusion over time. The medications may also be given in e.g. tablet form or in solution. Several appropriate medications and means for administration of the same are well known for treatment of PBC.

Uses

Also provided herein is the use of one or more biomarkers selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3 as a biological fluid biomarker for: predicting the level of response to UDCA in a test subject having primary biliary cholangitis; or selecting a treatment regimen for a test subject having primary biliary cholangitis.

Further provided herein is the use of one or more biomarkers selected from the group consisting of: CCL20, CXCL11, CXCL10, CXCL13, IL4RA, IL18R1, CD163, ACE2, CA5A, EpCAM, HAO1, DECR1, HAVCR1 and SCAMP3 as a biological fluid biomarker for determining the therapeutic effect of a treatment regimen for a test subject having primary biliary cholangitis.

Also provided herein is the use of one or more biomarkers selected from the group consisting of: CCL20, CXCL11, CXCL10, CXCL13, IL4RA, IL18R1, CD163, ACE2, CA5A, EpCAM, HAO1, DECR1, HAVCR1 and SCAMP3 as a biological fluid biomarker for monitoring the progression of primary biliary cholangitis in a test subject.

Details of the biomarkers (and particularly useful combinations of biomarkers), samples, methods, subjects, definitions etc. are provided elsewhere and apply equally to this aspect.

Methods for Monitoring PBC Progression

A method for monitoring the progression of primary biliary cholangitis in a test subject is also provided herein, the method comprising the steps of:
i) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EpCAM, HAO1, DECR1, HAVCR1 and SCAMP3;
ii) repeating step i) for the same test subject after a time interval; and
iii) comparing the biomarker levels identified in i) with the biomarker levels identified in ii),
wherein a change in the biomarker levels from i) to ii) is indicative of a change in primary biliary cholangitis progression in the test subject.

The term “change” refers in this context to a statistically significant difference. The difference (or change) may be an increase or decrease.

Typically, such monitoring methods are performed on subjects that have not yet been treated for PBC (i.e. they have not previously received PBC treatment). Such subjects are described as “treatment naïve” subjects herein.

However, such monitoring methods also encompass methods performed on subjects that have already been (or are being) treated for PBC. For example, the subject may have previously been diagnosed with PBC and may have previously received (or may be receiving) UDCA therapy. In this example, the methods described herein may be used to monitor PBC progression (e.g. relapse). The biomarkers that are described herein as particularly useful for distinguishing between PBC patients that will benefit from UDCA therapy (UDCA responders) and patients that will not benefit from UDCA therapy (UDCA non-responders). Accordingly, the methods described herein may be particularly useful when monitoring for non-response to UDCA treatment.

Monitoring the progression of PBC in a subject over time assists in the earliest possible identification of disease progression (e.g. a worsening in disease status or disease symptoms). Such monitoring naturally involves the taking of repeated samples over time. The method may therefore be repeated at one or more time intervals for a particular subject and the results compared to monitor the development, progression or improvement in PBC (and may also be used for example to monitor changes in the UDCA response status) in the subject over time, wherein a change in the amount of level of the one or more biomarker tested for in the biological fluid sample (e.g. blood) is indicative of a change in the progression of the PBC (and specifically a change in the UDCA response status) in the subject.

Disease progression (e.g. PBC progression, including not responding to UDCA therapy) may be indicated by an increase in the level of one or more of the specified biomarkers detected over time when the results of two or more time intervals are compared for the same subject. In other words, if the method is performed a plurality of times, disease progression may be indicated when the level of the one or more biomarker detected at the later time interval(s) is higher than that detected at the earlier time interval(s). An “increase” in the level of biomarker encompasses detection of biomarker at a later time interval when no biomarker was detected (i.e. it was not present at detectable levels) when the method was performed previously (i.e. at an earlier time interval) on the same subject (and an equivalent biological fluid sample type).

Suitable time intervals for monitoring disease progression can easily be identified by a person of skill in the art and will depend on the specific disease being monitored. As a non-limiting example, the method may be repeated at least every six months, or at least every year, or whenever clinically needed, i.e. in case of a significant change in PBC symptoms.

Details of the biomarkers (and particularly useful combinations of biomarkers), samples, methods, subjects, definitions etc. are provided elsewhere and apply equally to this aspect.

Methods for Determining the Therapeutic Effect of a Treatment Regimen for PBC

Methods provided herein may be used to monitor the effectiveness of a treatment for PBC. Monitoring the effectiveness of treatment may be useful for making treatment decisions.

A method for determining the therapeutic effect of a treatment regimen for a test subject having primary biliary cholangitis is therefore provided herein, the method comprising:
a) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EPCAM, HAO1, DECR1, HAVCR1 and SCAMP3;
b) repeating step a) using a biological fluid sample obtained from the test subject after treatment for a time interval; and
c) comparing the level of biomarker determined in step a) to that determined in step b), and identifying that the treatment regimen has a therapeutic effect if there is no increase in the level of the one or more biomarker after treatment, or if there is a decrease in the level of the one or more biomarker after treatment.

Typically, when a test subject is treated with any PBC treatment and is determined to have no increase in the level of the one or more biomarker after the treatment, or have a decreased level of the one or more biomarker after the treatment, the test subject is typically identified herein as a responder. Conversely, when a test subject is determined to have an increased level of the one or more biomarker after treatment, the test subject is typically identified herein as a non-responder.

For example, when a test subject is treated with UDCA and is determined to have no increase in the level of the one or more biomarker after UDCA treatment, or have a decreased level of the one or more biomarker after UDCA treatment, the test subject is typically identified herein as a UDCA responder. Conversely, when a test subject is determined to have an increased level of the one or more biomarker after UDCA treatment, the test subject is typically identified herein as a UDCA non-responder.

Similarly, when a test subject is treated with OCA and is determined to have no increase in the level of the one or more biomarker after OCA treatment, or have a decreased level of the one or more biomarker after OCA treatment, the test subject is typically identified herein as an OCA responder. Conversely, when a test subject is determined to have an increased level of the one or more biomarker after OCA treatment, the test subject is typically identified herein as an OCA non-responder.

In this context, a decrease of about 10% or more, about 20% or more, about 25% or more, about 30% or more, about 35%) or more, about 40% or more, about 45% or more, or about 50% or more may be indicative of a positive response to treatment.

Step a) may first be performed using a biological fluid sample that was obtained from the subject at a time point before the treatment regimen for PBC began. Alternatively, step a) may first be performed using a biological fluid sample that was obtained from the subject at the same time as commencing the treatment regimen, or at a time point after the treatment regimen for PBC began. The method can therefore be used to determine the therapeutic effect of a treatment regimen for PBC from the outset (i.e. from the start of the regimen) or from a time point after the treatment regimen has started (i.e. determining the therapeutic effect of a treatment regimen for PBC during the treatment regimen itself).

The method can also be useful as a screening tool for determining if specific regimens or treatment modalities have a therapeutic effect on PBC. The tested regimens or treatment modalities may be new regimens or treatment modalities, modified regimens or treatment modalities, or known regimens or treatment modalities that need further testing. In this context, a treatment modality is e.g. a drug or medicament that is useful or suspected to be useful in the treatment of PBC.

A treatment regimen may be identified as having a therapeutic effect if it results in a delay in disease progression or a delay in the development of symptoms (e.g. over a treatment period).

A treatment regimen may also be identified as having a therapeutic effect if it results in an improvement in disease status or symptoms (e.g. over a treatment period). Methods for determining if the treatment regimen has a therapeutic effect are well known in the art.

A treatment period refers to a time interval over which treatment occurs (e.g. 1 week, 2 weeks, 3 weeks, 1 month, 3 months, 6 months, 1 year, 3 years etc.).

As an example, an improvement in disease status or symptoms (e.g. over a treatment period) (e.g. improvement in PBC status or symptoms) may be indicated by a decrease in the level of one or more of the biomarkers of interest detected over time when the results of two or more time intervals are compared for the same subject. In other words, if the method is performed a plurality of times, an improvement in disease status may be indicated when the level of the biomarker(s) detected at the later time interval(s) is lower than that detected at the earlier time interval(s). A “decrease” in the level of biomarker encompasses no detection of the biomarker (i.e. it is not present at detectable levels) at a later time interval when the biomarker was detected when the method was performed previously (i.e. at an earlier time interval) on the same subject (and an equivalent biological fluid sample type).

An improvement in disease status or symptoms (e.g. over a treatment period) may also be indicated by stabilised levels of the one or more biomarker over time (compared to the level of the biomarker(s) observed in the absence of treatment over the equivalent time period, or compared to equivalent controls).

Suitable time intervals for monitoring an improvement in disease status or symptoms (e.g. during treatment of the subject) can easily be identified by a person of skill in the art and will depend on the specific form of disease being monitored. As a non-limiting example, the method may be repeated at least every six months, or at least every year, or at least every two years, or more frequently as required.

Details of the biomarkers, combinations, samples, methods steps, subjects, treatments, etc are provided elsewhere and apply equally to this aspect.

Methods of Treating PBC

A method for treating a subject having primary biliary cholangitis is also provided herein, the method comprising: administering an FXR agonist or a fibrate to a subject, wherein the subject is identified as in need of treatment for primary biliary cholangitis based on having, in a biological fluid sample, an increased level of one or more biomarker selected from the group consisting of: CCL20, CXCL11, CXCL10, CXCL13, CCL19, IL4RA, IL18R1, CD163, ACE2, CA5A, EPCAM, HAO1, DECR1, HAVCR1 and SCAMP3 compared to a threshold level or range, optionally wherein the FXR agonist is obeticholic acid. Optionally, the fibrate is selected from bezafibrate, fenofibrate or gemfibrozil.

Typically, the subject is undergoing or has previously undergone treatment with UDCA. Administration of the FXR agonist or fibrate is typically in combination with the UDCA treatment, but may also be without the UDCA treatment (i.e. administration of the FXR agonist or fibrate only).

A method for treating a subject having primary biliary cholangitis is further provided herein, the method comprising: administering UDCA to a subject, wherein the subject is identified as in need of treatment for primary biliary cholangitis based on having, in a biological fluid sample, an equivalent or decreased level of one or more biomarker selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3 compared to a threshold level or range. Typically, in such subjects, administration of UDCA is sufficient to treat the PBC (i.e. administration of additional second line agents such as an FXR agonist or a fibrate are not required).

Details of the biomarkers, combinations, samples, methods steps, subjects, treatments, etc. are provided elsewhere and apply equally to this aspect.

Kits and Assay Devices

Kits are also provided herein. The kits are suitable for carrying out one or more of the methods described herein. The kits include reagents suitable for determining levels of a plurality of analytes in a test sample (e.g., reagents suitable for determining levels of the biomarkers disclosed herein).

The kits described herein typically comprise:
(i) a detectably labelled agent that specifically binds to CCL20 protein; and
(ii) one or more of:
   (a) a detectably labelled agent that specifically binds to CXCL11 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Alternatively, the kit may comprise:
(i) a detectably labelled agent that specifically binds to CXCL11 protein; and
(ii) one or more of:
   (a) a detectably labelled agent that specifically binds to CCL20 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Alternatively, the kit may comprise:
(i) a detectably labelled agent that specifically binds to CXCL10 protein; and
(ii) one or more of:
   (a) a detectably labelled agent that specifically binds to CCL20 protein;
   (b) a detectably labelled agent that specifically binds to CXCL11 protein;
   (c) a detectably labelled agent that specifically binds to CXCL9 protein; and
   (d) a detectably labelled agent that specifically binds to CCL19 protein.

Alternatively, the kit may comprise:
(i) a detectably labelled agent that specifically binds to CXCL9 protein; and
(ii) one or more of:
   (a) a detectably labelled agent that specifically binds to CCL20 protein;
   (b) a detectably labelled agent that specifically binds to CXCL11 protein;
   (c) a detectably labelled agent that specifically binds to CXCL10 protein; and
   (d) a detectably labelled agent that specifically binds to CCL19 protein.

Alternatively, the kit may comprise:
(i) a detectably labelled agent that specifically binds to CCL19 protein; and
(ii) one or more of:
   (a) a detectably labelled agent that specifically binds to CCL20 protein;
   (b) a detectably labelled agent that specifically binds to CXCL11 protein;
   (c) a detectably labelled agent that specifically binds to CXCL10 protein; and
   (d) a detectably labelled agent that specifically binds to CXCL9 protein.

In any of the above examples, the kit may also comprise a detectably labelled agent that specifically binds to CXCL13 protein.

For example, the kit may comprise a plurality of distinct detectably labelled agents that specifically (independently) bind to CCL20 and CXCL11; CCL20 and CXCL10; CCL20 and CCL19; or CCL20 and CXCL9. The kit may comprise a plurality of distinct detectably labelled agents that specifically (independently) bind to CCL20, CXCL11 and CXCL10. Alternatively, kit may comprise a plurality of distinct detectably labelled agents that specifically (independently) bind to CCL20, CXCL11 and CCL19. Alternatively, the kit may comprise a plurality of distinct detectably labelled agents that specifically (independently) bind to CCL20, CXCL11 and CXCL9.

For example, the kit may comprise a plurality of distinct detectably labelled agents that specifically (independently) bind to CCL20, CXCL11, CXCL10 and CCL19. Alternatively, kit may comprise a plurality of distinct detectably labelled agents that specifically (independently) bind to CCL20, CXCL11, CCL19 and CXCL9. Alternatively, the kit may comprise a plurality of distinct detectably labelled agents that specifically (independently) bind to CCL20, CXCL11, CXCL10 and CXCL9.

For example, the kit may comprise a plurality of distinct detectably labelled agents that specifically (independently) bind to CCL20, CXCL11, CXCL10, CCL19 and CXCL9. Alternatively, kit may comprise a plurality of distinct detectably labelled agents that specifically (independently) bind to CCL20, CXCL11, CCL19 and CXCL9. Alternatively, the kit may comprise a plurality of distinct detectably labelled agents that specifically (independently) bind to CCL20, CXCL11, CXCL10 and CXCL9.

In a further example, the kit may comprise a plurality of distinct detectably labelled agents that specifically (independently) bind to CXCL11 and CXCL10; CXCL11 and CCL19; or CXCL11 and CXCL9. Alternatively, kit may comprise a plurality of distinct detectably labelled agents that specifically (independently) bind to CCL19 and CXCL9.

In any of the above examples, the kit may also comprise a detectably labelled agent that specifically binds to CXCL13 protein.

The kits described herein can take on a variety of forms. Typically, the kits will include reagents suitable for determining levels of a plurality of biomarkers disclosed herein in a sample.

Optionally, the kits may contain one or more control samples or references. Typically, a comparison between the levels of the biomarkers in the subject and levels of the biomarkers in the control samples or references is indicative of a clinical status. Also, the kits, in some cases, will include written information (indicia) providing a reference (e.g., pre-determined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

The components of the kit may be housed in a container that is suitable for transportation. Details on the biomarkers is given above and applies equally here.

The term “detectably labelled agent” refers to a binding partner that interacts (i.e. binds) specifically with the biomarker of interest and is also capable of being detected e.g. directly (such as via a fluorescent tag) or indirectly (such as via a labelled secondary antibody). The detectably labelled agent is therefore a selective binding partner for the biomarker of interest (and does not substantially bind to other proteins). Selective binding partners may include antibodies that selectively bind to one of the biomarker of interest.

As used herein, “specifically binds to CCL20” refers to selective binding of the CCL20 peptide. Under certain conditions, for example in an immunoassay as described herein, a binding partner that “specifically binds to CCL20” will selectively bind to this peptide and will not bind in a significant amount to other peptides. Thus the binding partner may bind to CCL20 with at least 10, 20, 30, 40, 50, 60, 70, 80, 90 or 100 fold more affinity than it binds to a control peptide. This definition also applies individually to each of the other biomarkers referred to herein.

In some examples the kits include the detectably labelled agent(s) on a continuous (e.g. solid) surface, such as a lateral flow surface. Alternatively, in examples comprising more than one detectably labelled agent, the detectably labelled agent(s) may be located in distinct (i.e. spatially separate) zones on a (e.g. solid) surface, such as a multiwall micro-titre plate (e.g. for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art). In one example, the kit comprises one or more of the following: a multi-well plate, ball bearing(s), extraction buffer, extraction bottle and a lateral flow device lateral flow device.

An assay device is also provided herein. The assay device is suitable for carrying out one or more of the methods described herein.

Typically, the device comprises a surface with at least two detectably labelled agents located thereon, wherein the at least two detectably labelled agents are:
(i) a detectably labelled agent that specifically binds to CCL20 protein; and
(ii) one or more of:
   (a) a detectably labelled agent that specifically binds to CXCL11 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Alternatively, the device may comprise a surface with at least two detectably labelled agents located thereon, wherein the at least two detectably labelled agents are:
(i) a detectably labelled agent that specifically binds to CXCL11 protein; and
(ii) one or more of:
   (a) a detectably labelled agent that specifically binds to CCL20 protein;
   (b) a detectably labelled agent that specifically binds to CXCL10 protein;
   (c) a detectably labelled agent that specifically binds to CXCL9 protein; and
   (d) a detectably labelled agent that specifically binds to CCL19 protein.

Alternatively, the device may comprise a surface with at least two detectably labelled agents located thereon, wherein the at least two detectably labelled agents are:
(i) a detectably labelled agent that specifically binds to CXCL10 protein; and
(ii) one or more of:
   (a) a detectably labelled agent that specifically binds to CCL20 protein;
   (b) a detectably labelled agent that specifically binds to CXCL11 protein;
   (c) a detectably labelled agent that specifically binds to CXCL9 protein; and
   (d) a detectably labelled agent that specifically binds to CCL19 protein.

Alternatively, the device may comprise a surface with at least two detectably labelled agents located thereon, wherein the at least two detectably labelled agents are:
(i) a detectably labelled agent that specifically binds to CXCL9 protein; and
(ii) one or more of:
   (a) a detectably labelled agent that specifically binds to CCL20 protein;
   (b) a detectably labelled agent that specifically binds to CXCL10 protein;
   (c) a detectably labelled agent that specifically binds to CXCL11 protein; and
   (d) a detectably labelled agent that specifically binds to CCL19 protein.

Alternatively, the device may comprise a surface with at least two detectably labelled agents located thereon, wherein the at least two detectably labelled agents are:
(i) a detectably labelled agent that specifically binds to CCL19 protein; and
(ii) one or more of:
   (a) a detectably labelled agent that specifically binds to CCL20 protein;
   (b) a detectably labelled agent that specifically binds to CXCL10 protein;
   (c) a detectably labelled agent that specifically binds to CXCL9 protein; and
   (d) a detectably labelled agent that specifically binds to CXCL11 protein.

In any of the above examples, the device may also comprise a detectably labelled agent that specifically binds to CXCL13 protein.

For example, the device may comprise a plurality of distinct detectably labelled agents that specifically (independently) bind to CCL20 and CXCL11; CCL20 and CXCL10; CCL20 and CCL19; or CCL20 and CXCL9. The device may comprise a plurality of distinct detectably labelled agents that specifically (independently) bind to CCL20, CXCL11 and CXCL10. Alternatively, device may comprise a plurality of distinct detectably labelled agents that specifically (independently) bind to CCL20, CXCL11 and CCL19. Alternatively, the device may comprise a plurality of distinct detectably labelled agents that specifically (independently) bind to CCL20, CXCL11 and CXCL9.

For example, the device may comprise a plurality of distinct detectably labelled agents that specifically (independently) bind to CCL20, CXCL11, CXCL10 and CCL19. Alternatively, device may comprise a plurality of distinct detectably labelled agents that specifically (independently) bind to CCL20, CXCL11, CCL19 and CXCL9. Alternatively, the device may comprise a plurality of distinct detectably labelled agents that specifically (independently) bind to CCL20, CXCL11, CXCL10 and CXCL9.

For example, the device may comprise a plurality of distinct detectably labelled agents that specifically (independently) bind to CCL20, CXCL11, CXCL10, CCL19 and CXCL9. Alternatively, device may comprise a plurality of distinct detectably labelled agents that specifically (independently) bind to CCL20, CXCL11, CCL19 and CXCL9. Alternatively, the device may comprise a plurality of distinct detectably labelled agents that specifically (independently) bind to CCL20, CXCL11, CXCL10 and CXCL9.

In a further example, the device may comprise a plurality of distinct detectably labelled agents that specifically (independently) bind to CXCL11 and CXCL10; CXCL11 and CCL19; or CXCL11 and CXCL9. Alternatively, device may comprise a plurality of distinct detectably labelled agents that specifically (independently) bind to CCL19 and CXCL9.

In any of the above examples, the device may also comprise a detectably labelled agent that specifically binds to CXCL13 protein.

The at least two detectably labeled agents may be located in separate zones on the surface. In other words, the at least two detectably labelled agents may be located in distinct (i.e. spatially separate) zones on a (e.g. solid) surface, such as a multiwell microtitre plate.

Detectably labelled agent(s) that specifically bind to the biomarker(s) of interest are described in detail elsewhere herein.

The assay device comprises a surface upon which the detectably labelled agents are located. Appropriate surfaces include a continuous (e.g. solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device described herein.

Appropriate assay device formats therefore include but are not limited to device formats suitable for performing any one of lateral flow, dot blot, ELISA, or surface plasmon resonance assays for detecting the presence, level or absence of the biomarker of interest.

Data Storage Aspects

The methods described herein may further include communication of the results or diagnoses (or both) to technicians, physicians or patients, for example. In certain examples, computers will be used to communicate results or diagnoses (or both) to interested parties, e.g., physicians and their patients.

In some examples, the results or diagnoses (or both) are communicated to the subject as soon as possible after the diagnosis is obtained. The results or diagnoses (or both) may be communicated to the subject by the subject's treating physician. Alternatively, the results or diagnoses (or both) may be sent to a subject by email or communicated to the subject by phone. A computer may be used to communicate the results or diagnoses by email or phone. In certain examples, the message containing results or diagnoses may be generated and delivered automatically to the subject using a combination of computer hardware and software which will be familiar to artisans skilled in telecommunications.

Companion Diagnostic

The methods, kits, assay devices and uses provided herein may be used as part of a companion diagnostic e.g. as part of an assay device which provides information that is essential for the safe and effective use of a corresponding drug or biological product (wherein the corresponding drug or biological product is for treating or preventing PBC, such as UDCA, an FXR agonist (e.g. OCA) or a fibrate (e.g. bezafibrate, fenofibrate or gemfibrozil)).

Examples of Applications of Method Results

The methods described herein can provide results which can then facilitate decisions as to the care of the subject. Examples are provided below.

Assay-Guided Therapy

The methods described herein can facilitate a clinician in making a treatment decision for the subject, e.g., whether the results of the method suggest the PBC subject may or may not benefit from therapeutic intervention (e.g. specifically from treatment with UDCA only, treatment with UDCA in combination with second line therapeutic agents such as an FXR agonist (e.g. OCA) or a fibrate (e.g. bezafibrate, fenofibrate or gemfibrozil), or treatment with therapeutic agents such as an FXR agonist (e.g. OCA) or a fibrate (e.g. bezafibrate, fenofibrate or gemfibrozil) without UDCA). For example, based on the method results, a therapy can be selected for the subject based on the likelihood s/he will benefit from UDCA treatment.

In addition, methods for assessing severity or extent of progression of PBC can be used for monitoring a subject over time and adjusting treatment and/or prioritizing the subject for liver transplant.

The method results can guide a clinician as to which therapy for treatment of PBC should be administered. For example, for a subject with elevated levels of the one or more biomarker of interest, the subject may be prioritized for treatment with an FXR agonist such as OCA, (e.g. in combination with UDCA). A subject with lower levels of the one or more biomarker of interest, may be treated with UDCA only instead.

In certain aspects, the method results can guide a clinician in adjusting therapy (e.g., whether or not to continue therapy (e.g., so as to avoid relapse), increase or decrease dose, change therapy regimen (e.g., from monotherapy to combination therapy, or from non-surgical therapy to surgical therapy) where the patient is not receiving adequate therapeutic benefit (e.g., the patient is not responding to therapy), and the like). Such methods of monitoring therapy are useful in guiding further treatment decisions, such as whether continued administration of a drug regimen indicated, or whether the patient should receive a liver transplant. The methods of monitoring therapy of the present disclosure may be used in combination with other methods for assessing whether a subject responds to therapy (is a “responder”) or is not exhibiting a sufficient therapeutically beneficial response (is as “non-responder”).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5 to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1: The Serum Proteome of Primary Biliary Cholangitis

### Subjects & Methods

### Study Design

The aim of the study was to explore the peripheral blood proteome of UDCA untreated and treated PBC to inform understanding of the biology of the disease and response and non-response to therapy, and to identify potentially clinically useful biomarkers of UDCA non-response. In the first, discovery phase of the study the serum proteomic profiles of informative subject groups from the UK-PBC patient cohort were assessed. These groups were treatment naïve PBC patients (i.e. newly presenting patients prior to the commencement of UDCA therapy) and PBC patients treated for a minimum of 12 months with UDCA therapy at optimal dose and stratified for UDCA response, and healthy community controls. In the second, confirmatory stage putative UDCA-response chemokine markers identified in the first phase were validated in a second, fully independent cohort of PBC patients stratified for UDCA response status recruited from the Newcastle clinical cohort.

### Subjects

In total, 526 PBC patients and 97 healthy controls participated in the study. PBC study participants in the discovery phase were all members of the UK-PBC Cohort (www.UK-PBC.com). This cohort was established to undertake studies of treatment efficacy in PBC and to understand, in particular, the biological basis of high-risk disease. The details of the UK-PBC patient cohort have been described in detail previously. This is a large, prospective national cross-sectional cohort of PBC patients with detailed clinical data collection. Within the cohort is a nested-sub cohort with additional biofluid sampling and banking to accompany the clinical data collection. The current study used samples from this sub-cohort. The confirmatory cohort were patients under clinical follow-up in Newcastle for PBC who were not participants in the UK-PBC nested cohort study. All diagnoses of PBC were made on the basis of the standard criteria used in clinical practice, namely presence of at least two out of the three key PBC characteristics of cholestatic liver biochemistry, serum anti-mitochondrial or PBC-specific anti-nuclear antibody at a titre of >1 in 40 and compatible or diagnostic features on liver biopsy (Hirschfield et al., 2018).

UDCA-Treated PBC: These were PBC patients who had been taking UDCA at a therapeutic dose (13-15 mg/kg/day) for greater than 12 months. Clinical parameters, including serum biochemistry were available both pre-treatment and after 12 months of UDCA therapy. This allowed all UDCA-treated patients to be assigned to responder and non-responder groups using current standard criteria including Paris 1 (Alanine Transaminase (ALT)>2× upper limit of normal [ULN], Alkaline Phosphatase (ALP)>3×ULN, Bilirubin>1×ULN) (Corpechot et al., 2008), Paris 2 (ALP and ALT both >1.5×ULN)(Corpechot et al., 2011) and POISE (ALP <1.67×ULN and bilirubin <1×ULN) (Nevens et al., 2016). UK-PBC risk score 10-year predicted survival was also calculated for UDCA-treated patients after therapy. Patients taking fibrates and or OCA were not included in the current study.

Treatment-Naïve PBC: These were newly presenting PBC patients prior to the commencement of UDCA or any other PBC-related therapy.

Healthy Controls: These were healthy community controls, matched for age to the treatment naïve PBC patients. This cohort was recruited via the NIHR Cambridge Bioresource specifically for comparison with the UK-PBC Nested Cohort and was utilised in the discovery phase of the study.

### Methods

Blood was collected under the approval of the Human Tissue Authority (HTA) by the UK-PBC tissue Bioresource with written informed patient consent obtained in accordance with research and ethics committee (REC) approval (14/NW/1146). Serum samples, once collected, were stored at −80° C. until assayed using the Olink Proteomics™ platform. 4 μl serum was analysed for 368 analytes from 4 distinct panels including Cardiovascular II &Ill, Inflammation and Oncology II (https://www.olink.com/). 356/368 analytes passed a quality control threshold of >25% detectability. Utilising Proximity Extension Assay (PEA) technology, antibodies labelled with oligonucleotides were hybridised to sample antigens, generating a barcode. In turn, this was amplified by microfluidic quantitative PCR with normalised protein expression (NPX) data generated by a ΔΔct calculation method.

For the confirmatory study, the inventors measured levels of informative chemokines identified in the discovery stage as being associated with UDCA non-response (CXCL9 [MIG], CXCL10 [IP-10], CXCL11 [1-TAC], CCL19 [MIP-3β] and CCL20 [MIP-3α]) using the Meso Scale Discovery® (MSD (Rockville, MD, USA) electro-chemiluminescence assay platform. Specifically, the inventors created a custom multi-PLEX panel for detecting the above analytes using the U-PLEX Custom Biomarker format. In order to retain uniformity and validity, the assay was performed exactly as per the manufacturer's instructions. Briefly, each of the biotinylated capture antibodies were covalently linked to specific spots within each well through use of unique linkers. Following a quenching step, plates were ready for sample application. Sera were thawed on ice and clarified by centrifugation at 18,000 g for 10 mins at 4° C. Sera were diluted 1:1 with supplied diluent and 25 μl per well added in duplicate. A standard analyte cocktail dose calibration curve was prepared and 25 μl per well added in duplicate. Plates were incubated for 2 hours at room temperature with shaking (200RPM). Following a wash step, SULFO-TAG™ conjugated detection antibody cocktail (50 μl) was added to all wells and plates incubated as before. After washing, read buffer was added (150 μl) and the plates read on the MSD Sector Imager 6000 instrument. The data were analysed using MSD Discovery Workbench v4.0. This software employs 4-parameter logistic (FourPL) calibration curve fitting from which sample values were derived. Each analyte had a lower limit of detection (LLOD) below all the sample values so all samples were in range and measurable. All sample values for all analytes are given as pg/ml.

### Analysis

Normalised protein expression (NPX) data were normalised via R statistical programming software and differential expression between patient cohorts: UDCA-treated PBC, Treatment naïve PBC and healthy controls (age-matched) was calculated. This is displayed as log fold change versus −log¹⁰p value (adjusted for multiple comparisons); all values were thresholded at p<0.05 and 1.5 fold change. P values were adjusted for multiple comparisons using the Benjamini Hochberg post-hoc method. Direct comparison of individual analyte NPX data was performed with an unpaired T-test using a Welch's correction (GraphPad Prism software 7.0). Multiple linear regression was performed on NPX dataset for correlations with ALP and UK-PBC ten year mortality/transplant risk score in the different PBC cohorts. P values were adjusted for multiple comparisons on a log scale vs fold change of NPX data. Protein-protein interaction was analysed using the STRING platform and the Gene Ontology (GO) classification. The chemokines identified as disease-associated in the discovery phase were used as descriptors of UDCA responder status in linear discriminant analysis to identify those chemokines that could be used to optimally discriminate between patient responder status. The inventors used a stepwise forward variable selection method that started by identifying the markers that separated the responders and non-responders most accurately. The model was then extended by including further variables depending on the Wilk's lambda. Wilk's lambda tests how well each marker contributes to the model's ability to discriminate between groups. Each marker was tested by putting it into the model and then taking it out again, generating a Wilks A statistic. The significance of the change in A was measured with an F-test; if the F-value was greater than the critical value for F with the sample sizes, then the marker was included in the model and the analysis repeated with the remaining cytokines. The selected markers (all chemokines) were then incorporated into a linear discriminant analysis so that discriminant functions could be abstracted and used to re-classify the original data into responder and non-responder. The error rate of classification was calculated (the proportion of cases incorrectly assigned to responder and non-responder groups). Since the data set had comparatively few cases, the inventors used a bootstrapping procedure to estimate the robustness of the final model. For this, the inventors randomly selected 90% of the case data and undertook the discriminant analysis on the subset and reclassified cases within it to responder and non-responder groups. The inventors repeated this 500 times and calculated the proportion of cases correctly classified and summarised the variation in the form of a histogram. There was an assumption that the data were multivariate normality in using linear discriminant analysis, so the inventors used logistic regression to assess the validity of the variables in the final model as predictors of responder/non-responder status. All analyses were undertaken in the R statistical programming language using the libraries KlaR and MASS.

### Bile Duct Ligation Model and Therapy

10-12 week old wild-type adult C57BL/6 mice underwent sham or BDL surgery. Mice were anaesthetized with isoflurane and the common bile duct was surgically ligated. Buprenorphine pain relief was administered post operatively via intraperitoneal injection and animals were maintained at 25° C. in heated cabinet for the duration of the study. Cholestatic liver disease was induced for up to 14 days in the first instance. OCA 0.03% w/w (30 mg/kg) (kindly provided by Intercept pharmaceuticals) or UDCA 0.5% (500 mg/kg) (Sigma) w/w was incorporated into the diet and mice were fed either OCA or UDCA containing diet ad libitum versus control diet prophylactically (3 days prior to BDL surgery) or therapeutically, from 3 days post-surgery.

### Results

The characteristics of the study cohorts are outlined in Table 1a (discovery cohort) and Table 1b (confirmatory cohort). Liver function tests were all normal in the healthy controls.

TABLE 1a
Clinical characteristics of the study cohort
for the proteomics discovery stage.

| | UDCA- | | Treatment- | | Healthy- | |
|---|---|---|---|---|---|---|
| | Treated PBC | | Naïve PBC | | Controls | |
| | (n = 416) | | (n = 68) | | (n = 97) | |
| Median Age | 63 | | 58 | | 64 | |
| Female (%) | 89 | | 82 | | 76 | |
| UDCA-Treatment (%) | 100 | | 0 | | 0 | |
| ALP @ 1 year (SD) | 178.2 | (121.5) | 226.5 | (193.1) | 70.9 | (22.2) |
| ALT @ 1 year(SD) | 37.6 | (28.9) | 53.1 | (34.7) | 19.7 | (8.6) |
| Bilirubin @ 1 year | 11.4 | (10) | 11.1 | (8.4) | 11.2 | (5.5) |
| (SD) | | | | | | |

TABLE 1b
Clinical characteristics of the study cohort for
the chemokine confirmatory stage of the study.

| | UDCA- | | |
|---|---|---|---|
| | Responder | UDCA-Non- | |
| | PBC | Responder | |
| | (n = 25) | (n = 17) | |
| Median Age | 65 | 56 | |
| Female (%) | 91 | 89 | |
| UDCA-Treatment (%) | 100 | 100 | |
| ALP @ 1 year (SD) | 80.9 (6.7) | 342.3 | (22.1) |
| ALT @ 1 year(SD) | 24.3 (4.0) | 81.7 | (11.9) |
| Bilirubin @ 1 year (SD) | 6.6 (0.5) | 15.6 | (4.4) |

### Discovery Proteomics Study

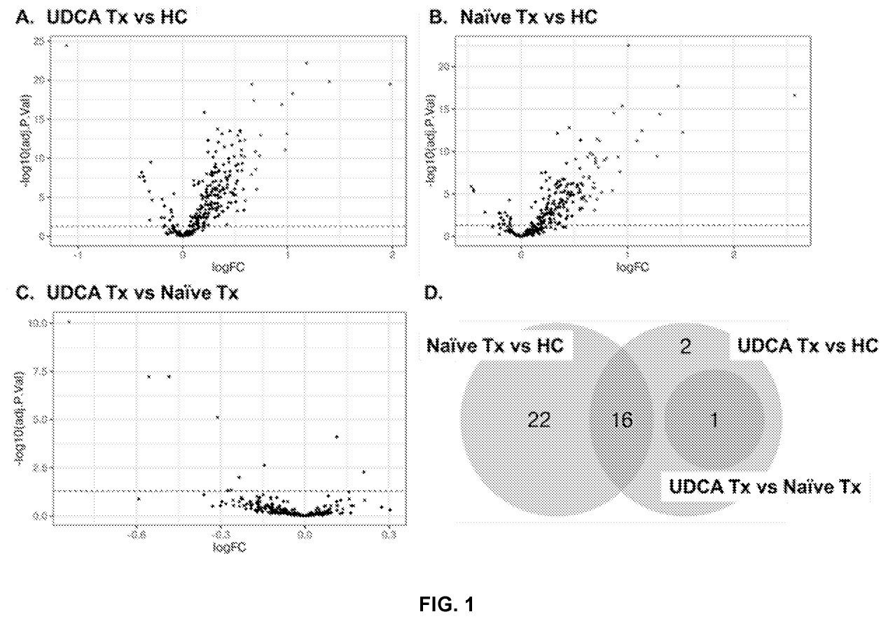

Initially, the inventors explored and compared the inflammatory proteome in the treatment-naïve PBC patients, the UDCA-treated patients and healthy controls (FIG. 1 A-D). A distinct PBC disease ‘profile’ in terms of the protein signature in the serum was seen when treatment-naïve PBC patients were compared with the healthy controls. The proteins significantly over-expressed in PBC are detailed in Table 2. The inflammatory proteome in the UDCA-treated patients was almost identical to the treatment-naïve patients, with only a single protein with a different expression pattern between UDCA-treated and treatment-naïve patients (Epcam). The extent to which analytes are differentially regulated in a similar manner in both groups of PBC patients (FIG. 1D) would suggest that UDCA treatment does not substantially change the nature of the serum proteome.

TABLE 2
Proteins showing significant differential expression between
study groups in the discovery proteomics stage of the study.

| Naïve Tx vs HC | | | UDCA Tx vs Naïve Tx | | |
|---|---|---|---|---|---|
| Protein | log | | Protein | log | |
| analyte | Change | Fold | analyte | Change | Fold |
| HAO1 | 2.57 | | Ep-CAM | −0.84 | |
| CCL20 | 1.52 | | | | |
| CCL19 | 1.48 | | | | |
| CXCL9 | 1.30 | | | | |
| CA5A | 1.28 | | | | |
| CXCL10 | 1.14 | | | | |
| CXCL11 | 1.09 | | | | |
| C′D40 | 1.01 | | | | |
| 5-NT | 0.95 | | | | |
| SCAMP3 | 0.93 | | | | |
| DECR1 | 0.91 | | | | |
| CXCL13 | 0.87 | | | | |
| ACE2 | 0.86 | | | | |
| TNFRSF6B | 0.80 | | | | |
| VIM | 0.78 | | | | |
| IL8 | 0.76 | | | | |
| TNFRSF4 | 0.74 | | | | |
| CXCL6 | 0.72 | | | | |
| EN-RAGE | 0.71 | | | | |
| MetAP 2 | 0.71 | | | | |
| XCL1 | 0.70 | | | | |
| IL-18R1 | 0.70 | | | | |
| IL-1ra | 0.70 | | | | |
| AP-N | 0.70 | | | | |
| AZU1 | 0.69 | | | | |
| NEMO | 0.69 | | | | |
| GDF-15 | 0.69 | | | | |
| IL-4RA | 0.68 | | | | |
| IL16 | 0.67 | | | | |
| TNFRSF9 | 0.65 | | | | |
| CCL3 | 0.65 | | | | |
| IL-12B | 0.64 | | | | |
| CDCP1 | 0.64 | | | | |
| CD163 | 0.63 | | | | |
| CCL3 | 0.61 | | | | |
| IL6 | 0.60 | | | | |
| TXLNA | 0.59 | | | | |

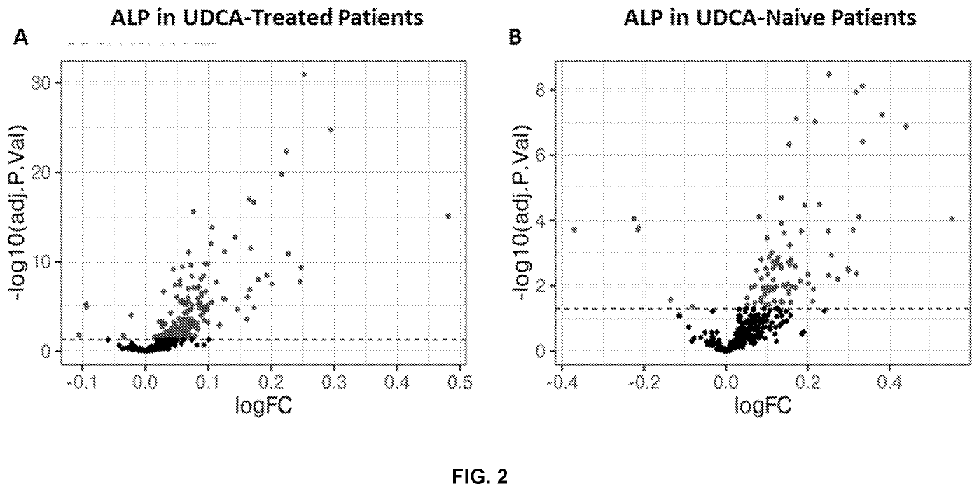

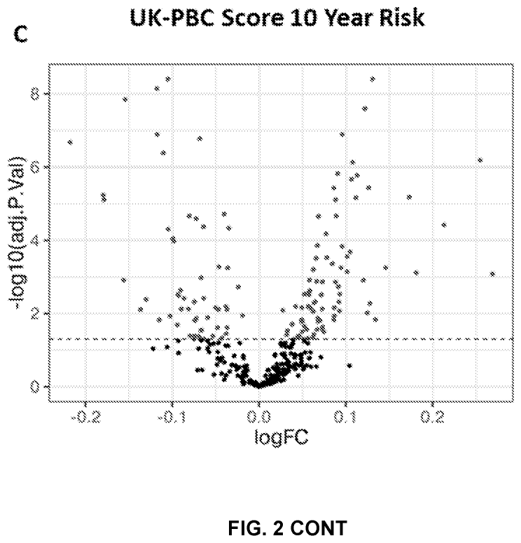

The inventors next went on to explore the relationship between the PBC proteome and both alkaline phosphatase levels (FIGS. 2A & B) and UK-PBC score (10-year predicted risk of death or transplant; FIG. 2C). 184 analytes in the UDCA-treated cohort and 92 analytes in the treatment-naïve cohort showed significant correlation with ALP levels. This finding suggests that ALP levels are associated with disease mechanistic markers in both the UDCA-treated and treatment-naïve states. Similarly, in the UDCA-treated patient cohort, the UK-PBC risk score 10-year risk correlated significantly with 138 analytes validating the score as a mechanistically relevant prognostic tool in PBC. A similar pattern of associations was seen also for the 5- and 15-year predicted mortality/transplant rates (data not shown).

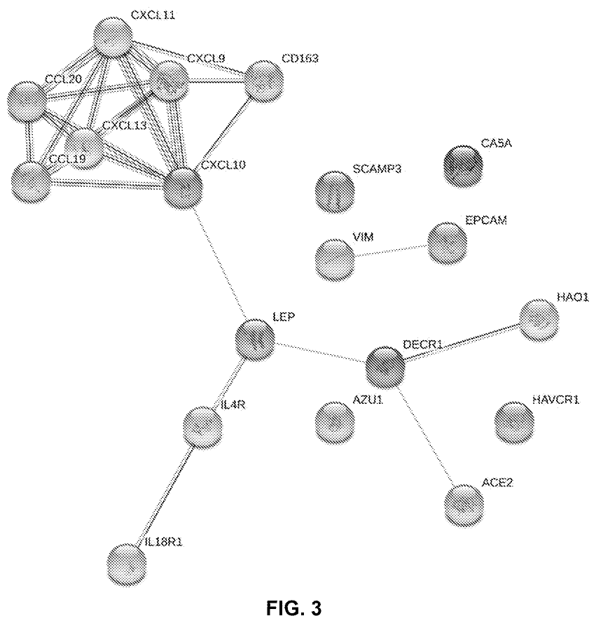

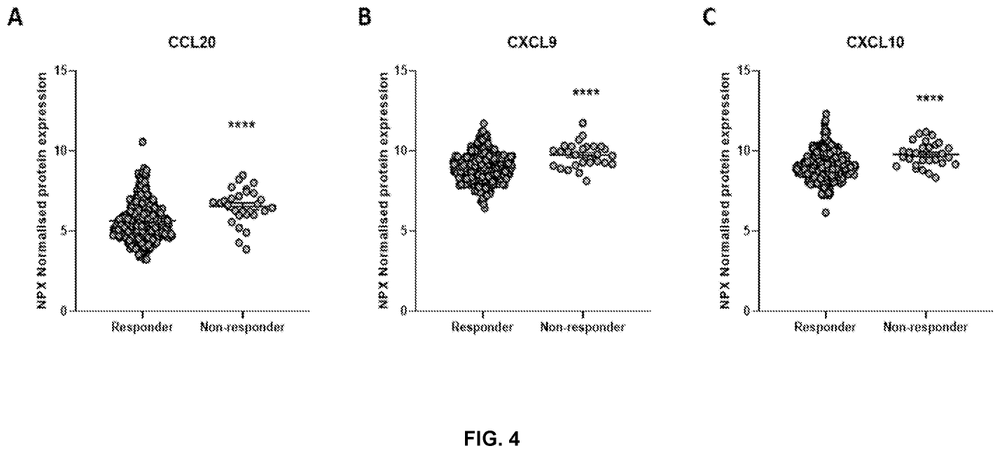

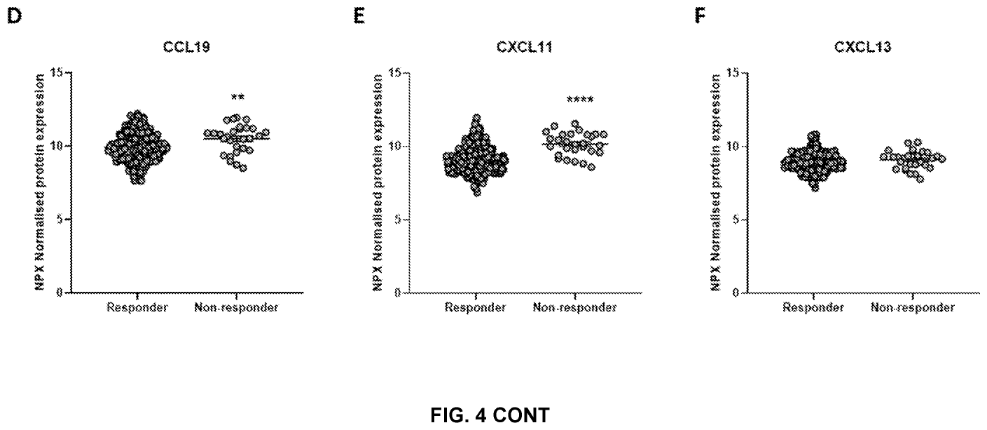

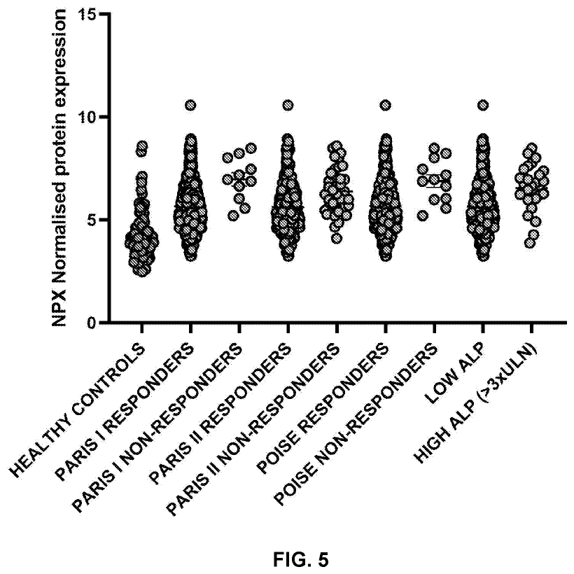

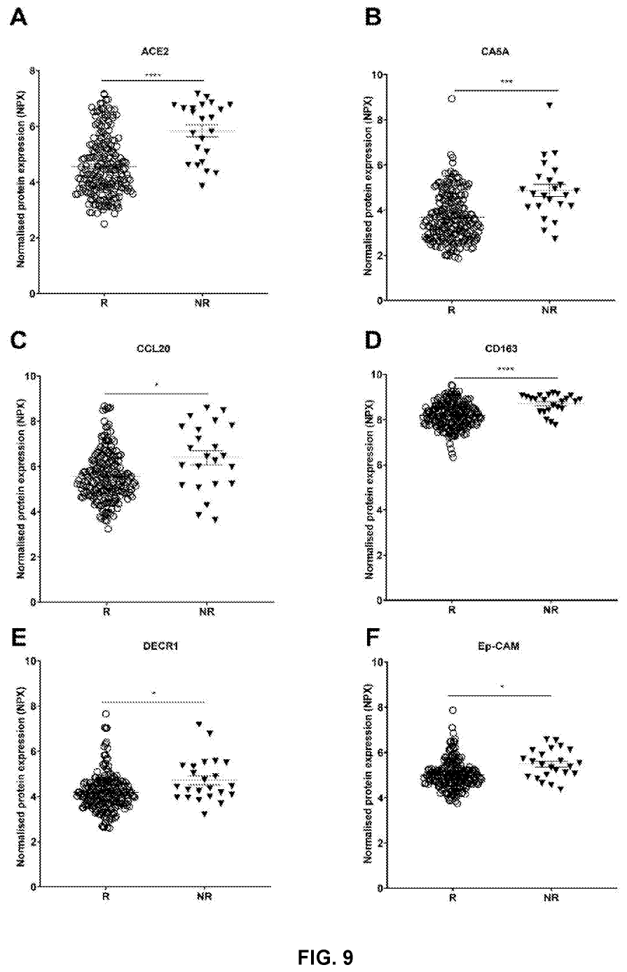

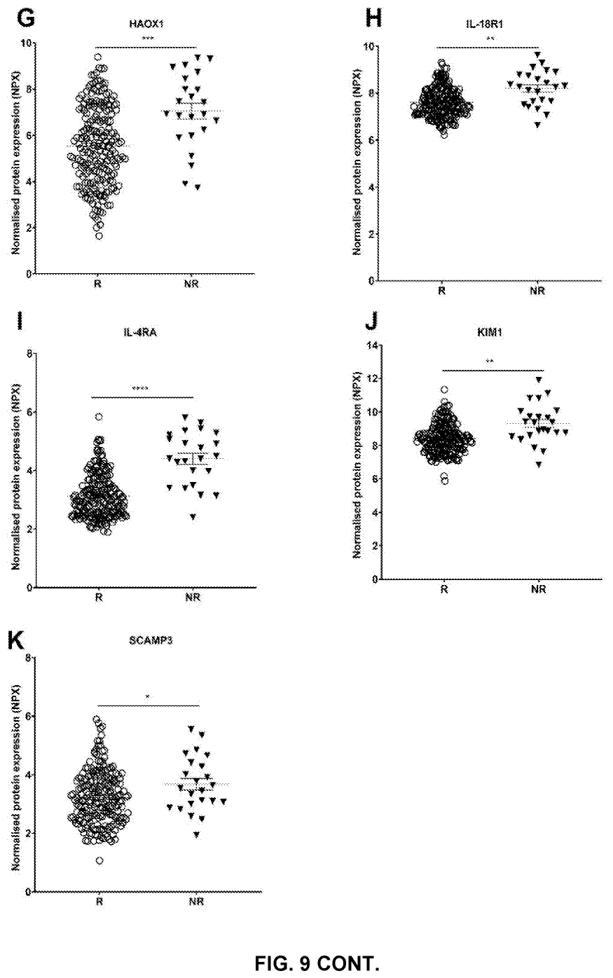

The inventors next explored the individual components of the PBC proteome. Significant protein-protein interaction was observed amongst the proteins significantly over-expressed in the treatment-naïve patients (PPI enrichment p value <10⁻¹⁶), suggesting a functional expression pattern. The greatest enrichment was seen for cytokine-mediated signalling pathways, inflammatory response and leucocyte chemotaxis gene ontology (GO)-terms (Table 3a, Table 3b). Comparison of the UDCA-treated PBC patients and the healthy controls demonstrated 19 proteins that remained significantly elevated despite UDCA therapy, with significant pathway enrichment particularly around chemotaxis where 6 inter-related chemokines were significantly elevated despite UDCA treatment (CCL19, CCL20, CXCL9, CXCL10, CXCL11 and CXCL 13; FIG. 3, Table 3c; see also FIG. 9). Of these 6 chemokines still over-expressed in PBC after UDCA-treatment (FIG. 4). The data in FIG. 4 are for treatment response assessed using Paris 1 criteria, however this effect remained true regardless of the UDCA response criteria used (FIG. 5). In all cases over-expression was seen in the non-responders. No proteins were, in contrast, significantly elevated in the UDCA-responders compared to the non-responders.

TABLE 3a
Functional enrichments in the treatment-naïve PBC protein
network. Data are shown for the 10 gene ontology (GO) biological
processes showing the greatest enrichment for each.

| | | False Discovery |
|---|---|---|
| GO-Term | Description | Rate |
| 0019221 | Cytokine-mediated signaling pathway | 3.6 × 10⁻²² |
| 0006954 | Inflammatory response | 7.9 × 10⁻²⁰ |
| 0030595 | Leucocyte chemotaxis | 9.1 × 10⁻¹⁸ |
| 0006952 | Defense response | 4.2 × 10⁻¹⁷ |
| 0006955 | Immune response | 1.9 × 10⁻¹⁶ |
| 0051707 | Response to other organism | 4.0 × 10⁻¹⁶ |
| 0050921 | Positive regulation of chemotaxis | 6.8 × 10⁻¹⁶ |
| 0032496 | Response to Ips | 2.2 × 10⁻¹⁵ |
| 0070098 | Chemokine-mediated signaling pathway | 8.4 × 10⁻¹⁵ |
| 0002687 | Regulation of signaling receptor activity | 1.5 × 10⁻¹⁴ |

TABLE 3b
Functional enrichments in the UDCA-treated protein network
in the discovery proteomics stage of the study. Data
are shown for the 10 gene ontology (GO) biological processes
showing the greatest enrichment for each.

| | | False Discovery |
|---|---|---|
| GO-Term | Description | Rate |
| 0050900 | Leucocyte migration | 3.4 × 10⁻⁹ |
| 0048247 | Lymphocyte chemotaxis | 3.4 × 10⁻⁹ |
| 0031640 | Killing of other organism | 3.4 × 10⁻⁹ |
| 0006954 | Inflammatory response | 3.4 × 10⁻⁹ |
| 0030595 | Leucocyte chemotaxis | 6.2 × 10⁻⁹ |
| 0019730 | Antimicrobial humoral response | 9.1 × 10⁻⁹ |
| 0070098 | Chemokine-mediated signaling pathway | 1.4 × 10⁻⁸ |
| 0019221 | Cytokine-mediated signaling pathway | 1.6 × 10⁻⁸ |
| 0002687 | Positive regulation of leucocyte migration | 1.6 × 10⁻⁷ |
| 0002690 | Positive regulation of leucocyte | 1.3 × 10⁻⁶ |
| | chemotaxis | |

TABLE 3c
Markers Significantly Elevated in UDCA Non-Responders Compared
to Responders. The first 5 markers were also validated in
a second cohort (see confirmatory chemokine study below).

| | |
|---|---|
| CCL20 | p < 0.0001 |
| CXCL11 | p < 0.0001 |
| CXCL10 | p < 0.0001 |
| CXCL9 | p < 0.0001 |
| CCL19 | p < 0.001 |
| IL4RA | p < 0.0001 |
| IL18R1 | p < 0.0001 |
| CD163 | p < 0.0001 |
| ACE2 | p < 0.0001 |
| CA5A | p < 0.0001 |
| EPCAM | p < 0.0001 |
| HAO1 | p < 0.0001 |
| DECR1 | p < 0.0001 |
| HAVCR1 | p < 0.0001 |
| SCAMP | p < 0.001 |

### Confirmatory Chemokine Study

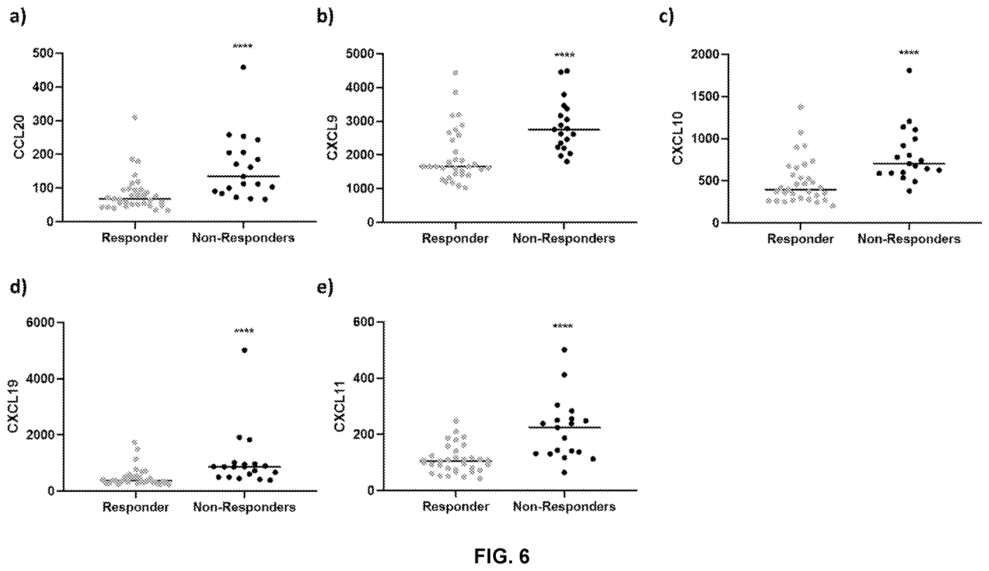

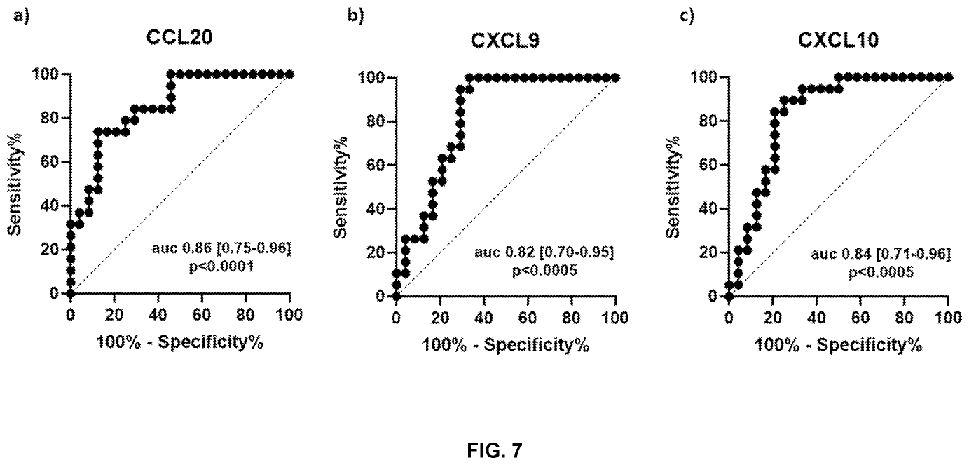

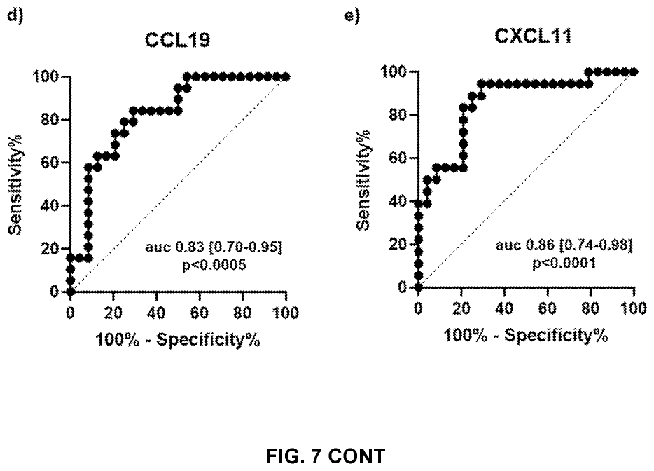

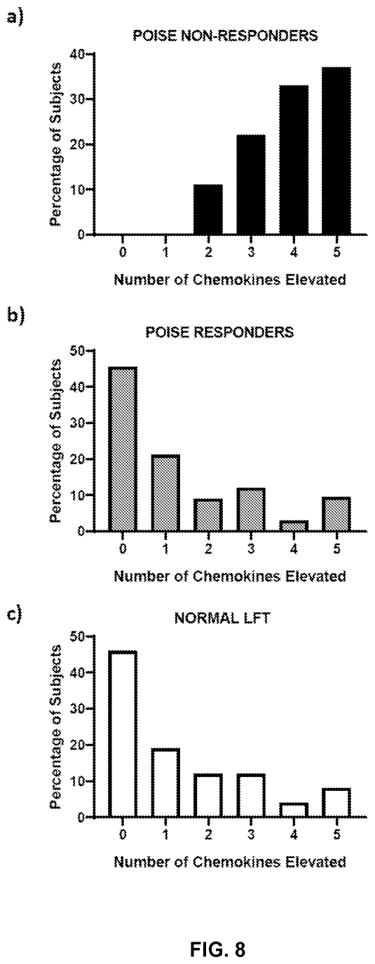

The inventors next studied the panel of 5 chemokines identified in the proteomics phase of the study as being associated with UDCA non-response in a second PBC patient cohort stratified for UDCA response and non-response using a multiplex assay system. All 5 chemokines again had significantly higher levels in UDCA non-responders compared to responders (FIG. 6), confirming the original observation. Furthermore, the level of each of the 5 chemokines was, individually, highly predictive of UDCA non-response/response status (FIG. 7). The inventors defined significant elevation of a chemokine based on the range of values seen in the key UDCA non-responder group (the group in which each chemokine is significantly elevated). The inventors defined a significant elevation as one greater than the lower 95% CI for the non-responders. The UDCA non-responders had significantly greater numbers of abnormal chemokines than the UDCA responders (FIG. 8a & b; Chi square 25.5, p=0.0001). The majority of non-responders had elevation in either 4 or 5 of the chemokines. Interestingly, over 50% of UDCA responders also had elevation of one or more chemokine (FIG. 8b). This also remained true for the group of UDCA responders who had normalised their liver function tests (LFTs; FIG. 8c).

Finally, the inventors went on to explore the capacity of chemokine elevation to act as a predictive mechanistic marker for identification of UDCA non-response status using linear discriminant analysis in the confirmatory study cohort. Wilks lambda values for each of the chemokines, and the significance of their discriminating power for separating responders and non-responders, are shown in Table 4. Only 3 of the chemokines were good discriminators, and one of these, CXCL9, did not lead to a significant increase in discrimination when added sequentially to a model that included CCL20 and CXCL11. Re-classification of the original confirmatory study data set led to 40 patients (87% of the cases) being correctly classified, with 2 patients predicted to be non-responders that were responders and 4 predicted to be responders that were not. The classification status is based on use of discriminant functions that effectively find a line in multivariate space that separates the groups. In the two groups, case values on this line below zero constitute membership of the non-responder group and values above zero predicted membership of the responder group. The bootstrapped discriminant analyses based on 500 repeat random samples of the data set, gave a mean percentage correctly classified as 85.1% (SD=2.3), suggesting that this is a robust classifier.

TABLE 4
Wilks lambda and discrimination between responder and non-responder
status for chemokines in the confirmatory chemokine study. Note
CXCL9 was non-significant at p > 0.05 and are hence excluded
from the final linear discriminant analysis (LDA) model.

| | Wilks | F | P | F | P |
|---|---|---|---|---|---|
| Chemokine | Lambda | Statistic | value | difference | difference |
| CCL20 | 0.689 | 19.85 | 5.68E−0.5 | 19.85 | 5.68E−0.5 |
| CXCL11 | 0.518 | 19.99 | 7.29E−07 | 14.17 | 4.91E−0.4 |
| CXCL9 | 0.498 | 14.13 | 1.64E−0.6 | 1.74 | 1.94E−0.1 |

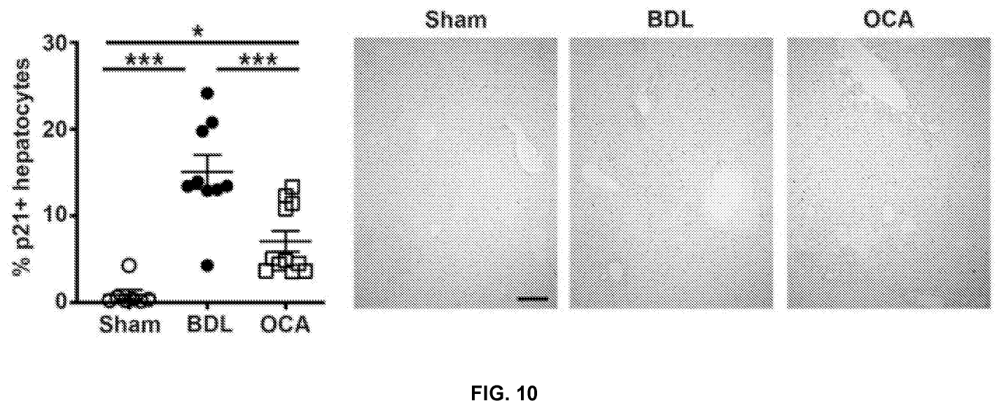

The inventors also explored whether administration of either the first-line therapy Ursodeoxycholic acid (UDCA) or the second-line therapy Obeticholic acid (OCA), currently licensed for the treatment of PBC, impacted on hepatocyte senescence in BDL mice treated with UDCA or OCA. Hepatocyte senescence detected by p21 staining and ductular DNA damage (γH2Ax) were comparable between BDL and UDCA treated animals suggesting that UDCA is not anti-senescent (data not shown). In marked contrast, both hepatic senescence and ductular DNA damage was attenuated in the liver of prophylactic OCA treated BDL mice (FIG. 10).

### Discussion

In this study, the inventors utilised two independent cohorts of deeply phenotyped PBC patients, including treatment-naïve patients and UDCA-treated patients stratified by their therapy response status, to characterise the serum proteome in the disease. The inventors aim was to explore the biological basis of non-response to UDCA in order to better understand how to identify and treat higher risk, therapy non-responding patients. This is the first large-scale study of serum proteomics in PBC with over 500 subjects in the discovery and confirmatory phases. The findings begin to shed light on the mechanisms underpinning incomplete response to UDCA in PBC.

The inventors' first observation is that treatment-naïve PBC patients show elevation of a series of inflammatory protein markers in their serum when compared to healthy community controls. This supports the idea that there is a significant inflammatory/immune component to PBC pathogenesis. Elevations of these proteins correlates with both alkaline phosphatase levels, pre- and post-UDCA therapy, and with the UK-PBC risk score predicted 10-year mortality post-UDCA therapy. To date, approval of second-line therapy in PBC has been on the basis of significant improvement in the biochemical marker alkaline phosphatase. Although alkaline phosphatase is predictive of disease risk in clinical practice, it is a surrogate marker at best and the lack of an apparent mechanistic link to the disease has weakened its value in the eyes of regulatory bodies. Here, the observation of a clear correlation between components of the PBC proteome and levels of serum alkaline phosphatase (as well as with the composite UK-PBC risk score) supports a direct link between alkaline phosphatase and underpinning immune and inflammatory processes and gives additional confidence in its use as a surrogate marker in clinical trials.

Comparison of the proteome in treatment naïve and UDCA-treated patients in the discovery phase identified only a single protein (EpCAM) that was normalised by UDCA treatment. The other components of the PBC proteome were reduced in expression level but remained present at significantly elevated levels after UDCA therapy. This finding suggests that UDCA treatment modifies, but typically does not resolve, disease-associated inflammatory processes in PBC.

A significant element of the untreated PBC proteome, persisting even after UDCA, is a network of chemokines (CCL20, CCL19, CXCL9, CXCL10, CXCL11 and CXCL13). CXCL9,-10 and -13 have been previously shown to be elevated in PBC patients, and not normalised by UDCA therapy; the inventors' findings confirm these earlier observations (Chuang et al., 2005; Manousou et al., 2013). In particular, the levels of the individual CXCL chemokines were strongly correlated in the current study (data not shown), probably reflecting a shared regulation in the form of IL-27 (Basset et al., 2015). Interestingly, the recent international meta-analysis of the genetic basis of PBC has identified the IL-27 pathway as a PBC susceptibility pathway (Cordell et al., 2015). Significant upregulation of CXCL9, CXCL10 and CCL19 has also been described in a murine PBC model, with levels being reduced by IL-22 therapy. CCL20, the gene encoding which was also identified as a susceptibility locus in PBC in the recent meta-analysis (Cordell et al., 2015), is released by injured biliary epithelial cells (BEC) in PBC (Harada et al., 2011; Oo et al., 2012).

In this study the inventors demonstrate, and then confirm in a second population, that levels of the chemokines CCL20, CCL19, CXCL9, CXCL10, CXCL13 and CXCL11 are significantly higher in UDCA non-responders than UDCA responders. This effect was the same regardless of the UDCA response criteria used. A key aspect of BEC injury that drives CCL20 release in particular is senescence (Etherington et al., 2019). BEC senescence has been shown to be a key adverse event in PBC pathogenesis, linked to both UDCA non-response and high risk of disease progression (Sasaki et al., 2020; Hardie et al., 2016). In a transcriptomic and immuno-histochemical study of the baseline liver biopsies of patients who went on to be non-responders to UDCA and show disease progression, duct injury including senescence as denoted by p21 expression, was present at disease outset, with CCL20 transcription upregulated (Hardie et al., 2016). In contrast, CCL20 transcription was not upregulated in liver biopsies of low risk patients at presentation and BEC senescence was absent. CCL20 appears to play a key role in the epithelial targeting of CCR6 expressing Th17 and Tc17 T-cells, strongly implicated in the immune-pathogenesis of PBC (Yang et al., 2014).

Therefore, there appears to be an important, bi-directional cross-talk between immune cells and senescent BEC, with CCL20 playing a key role (Jeffery et al., 2019). Far from being passive targets of immune injury in PBC, the BEC (especially in the context of cholestasis-induced senescence) appear to be complicit in shaping and localising the immune response that contributes to their injury. The apparent role of the BEC in modifying and localising T-cell reactivity may help to explain the importance of interface hepatitis as an adverse marker in high risk, UDCA non-responsive disease (Corpechot et al., 2008). The ongoing elevation of chemokines in UDCA responding patients, and the presence of at least one elevated UDCA non-responder associated chemokine in over 50% of even the responder group who have normalised their LFTs, points to the effect of UDCA in terms of disease modification being incomplete in a large proportion of patients.

The inventors' observation that measurement of just CCL20 and CXCL11 is sufficient to identify UDCA non-responders with a high degree of accuracy (addition of the other chemokines to a predictive model does not further improve accuracy) points to a potentially useful mechanistic biomarker for high risk PBC. The replication of the chemokine association with UDCA non-response in a second cohort suggests that this key finding is robust.

In conclusion, the inventors have demonstrated that PBC has a characteristic inflammatory proteome. Strikingly, this is only modified to a modest degree by treatment with standard therapy UDCA with, in particular, a prominent ongoing chemokine response even on full dose UDCA. This suggests a mechanism for UDCA non-response, potentially invoking ongoing BEC stress/senescence despite UDCA therapy.

### Example 2

### Second Validation Cohort

Further validation of the markers CCL20 and CXCL11 was undertaken on a cohort of 197 further patients with an established diagnosis of PBC and who are established on UDCA therapy (90.5% female). Of these, 154 were responders to UDCA as defined using the POISE criteria (ALP<1.67×uln and bilirubin <1×uln; the standard UK clinical response criteria) whilst 43 were non-responders. The characteristics of the study group are in the table below.

TABLE 5
characteristics of the study group

| | Whole Group | Responder | Non-Responder |
|---|---|---|---|
| Age | 61.6 ± 11.1 | 63.8 ± 9.8 | 54.4 ± 12.7 |
| ALP | 174 ± 144 | 119 ± 41 | 374 ± 196 |
| ALT | 40 ± 36 | 29 ± 16 | 82 ± 53 |
| Bil | 11.7 ± 19.0 | 7.9± 4.4 | 25.0 ± 37.1 |

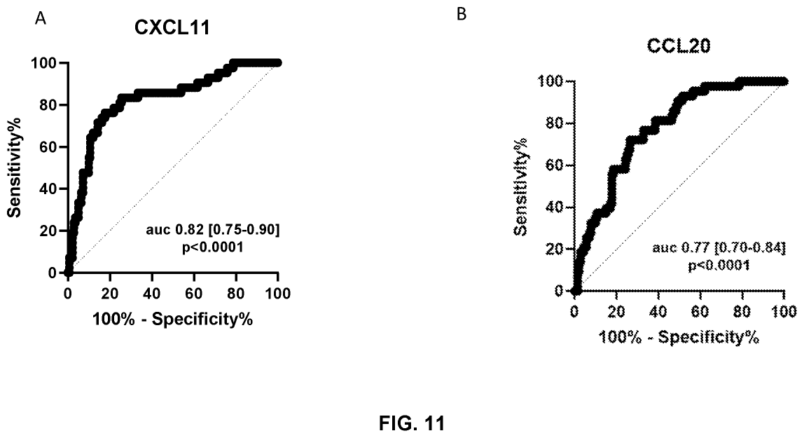

Both CXCL11 and CCL20 were again strongly predictive of non-response status, with CXCL11 out-performing CCL20 (see FIGS. 11 A and B). Using the composite diagnostic there was a ppv of 0.89 and an npv of 0.81 for the optimal cut-off.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### EXAMPLE 1 REFERENCES

1. Tinmouth J, Lee M, Wanless I R, Tsui F W L, Inman R, Heathcote E J. Apoptosis of biliary epithelial cells in primary biliary cirrhosis and primary sclerosing cholangitis. Liver 2002; 22:228-34.
2. Sasaki M, Miyakoshi M, Sato Y, Nakanuma Y. Modulation of the microenvironment by senescent biliary epithelial cells may be involved in the pathogenesis of primary biliary cirrhosis. J Hepatol 2010; 53:318-25.
3. Cordell H J, Han Y, Mells G F, et al. International genome-wide meta-analysis identifies new primary biliary cirrhosis risk loci and targetable pathogenic pathways. Nat Commun 2015; 6:8019.
4. de Graaf K, Lapeyre G, Guilhot F, et al. NI-0801, an anti-CXCL10 antibody, in patients with primary biliary cholangitis and an incomplete response to ursodeoxycholic acid. Hepatol Comm 2018; 2:492-503.
5. Hirschfield G M, Dyson J K, Alexander G J M, et al. The British Society of Gastroenterology/U K-PBC primary biliary cholangitis treatment and management guidelines. Gut 2018; 67:1568-94.
6. Corpechot C, Abenavoli L, Rabahi N, et al. Biochemical response to ursodeoxycholic acid and long-term prognosis in primary biliary cirrhosis. Hepatology 2008; 48:871-7.
7. Chuang Y H, Lian Z X, Cheng C M, et al. Increased levels of chemokine receptor CXCR3 and chemokines IP-10 and MIG in patients with primary biliary cirrhosis and their first degree relatives. J Autoimmun 2005; 25:126-32.
8. Manousou P, Kolios G, Drygiannakis I, et al. CXCR3 axis in patients with primary biliary cirrhosis: a possible novel mechanism of the effect of ursodeoxycholic acid. Clin Exp Immunol 2013; 172:9-15.
9. Basset L, Chevalier S, Danger Y, et al. Interleukin-27 and IFN-gamma regulate the expression of CXCL9, CXCL10 and CXCL11 in hepatitis. J Mol Med (Berl) 2015; 93:1355-67.
10. Harada K, Shimoda S, Ikeda H, et al. Significance of periductal Langerhans cells and biliary epithelial cell-derived macrophage inflammatory protein-3a in the pathogenesis of primary biliary cirrhosis. Liver Int 2011; 31:245-53.
11. Oo Y H, Banz V, Kavanagh D, al e. CXCR3-dependent recruitment and CCR6-mediated positioning of Th-17 cells in the inflamed liver. J Hepatol 2012; 57:1044-51.
12. Etherington R E, Millar B J M, Innes B A, Jones D E J, Kirby J A, Brain J G. Bile acid receptor agonists in primary biliary cholangitis: Regulation of the cholangiocyte secretome and downstream T cell differentiation. Faseb Bioadvances 2019:1-12.
13. Sasaki M, Sato Y, Nakanuma Y. Increased p16|NK4a-expressing senescent bile ductular cells are associated with inadequate response to ursodeoxycholic acid in primary biliary cholangitis. J Autoimmun 2020; In press.
14. Hardie C, Green K, Jopson L, et al. Molecular Stratification of High-Risk Primary Biliary Cholangit. E-Bioscience 2016; In press.
15. Yang C Y, Ma X, Tsuneyama K, et al. IL-12/Th1 and IL-23/Th17 biliary microenvironment in primary biliary cirrhosis: implications for therapy. Hepatology 2014; 59:1944-53.
16. Jeffery H C, Hunter S, Humphreys E H, et al. Bidirectional cross-talk between biliary epithelium and Th17 cells promotes local Th17 expansion and bile duct proliferation in biliary liver diseases. J Immunol 2019; 203:1151-9.
17. Carbone M. et. al., Hepatology. 2016 March; 63(3):930-50
18. Kaplan et al. The New England Journal of Medicine, 2005. 353(12): p. 1261-73.
19. Heitzer, E., Haque, I. S., Roberts, C. E. S. et al. Current and future perspectives of liquid biopsies in genomics-driven oncology. Nat Rev Genet 20, 71-88 (2019).
20. Corpechot C, Chazouilleres O, Poupon R. Early primary biliary cirrhosis: biocehmical response to treatment and prediction of long-term outcome. J Hepatol 2011; 55:1361-7.
21. Nevens F, Andreone P, Mazzella G, et al. A Placebo-Controlled Trial of Obeticholic Acid in Primary Biliary Cholangitis. N Engl J Med 2016; 373:631-43.

1. A method for predicting the level of response to ursodeoxycholic acid (UDCA) in a test subject having primary biliary cholangitis, the method comprising the steps of:
a) determining the level of one or more biomarker in a biological fluid sample from the test subject, wherein the one or more biomarker is selected from the group consisting of: CCL20, CXCL11, CXCL10, CXCL13, CCL19, IL4RA, IL18R1, CD163, ACE2, CA5A, EpCAM, HAO1, DECR1, HAVCR1 and SCAMP3;
b) comparing the level of the one or more biomarker with a threshold level or range; and
c) predicting that:
i) a test subject having a decreased level of the one or more biomarker compared to the threshold level or range is a UDCA responder; and
ii) a test subject having an increased level of the one or more biomarker compared to the threshold level or range is a UDCA non-responder.

2. The method of claim 1, comprising determining the level of at least two, three, or four of the biomarkers, optionally wherein the at least two, three, or four biomarkers are selected from the group consisting of: CCL20, CXCL11, CXCL10 and CCL19.

3. (canceled)

4. The method of claim 2, wherein the at least two biomarkers comprise CCL20 and CXCL11.

5. The method of claim 1, further comprising determining the level of CXCL9 in the biological fluid sample.

6. The method of any of claim 1, wherein, for test subjects that are predicted to be UDCA non-responders, the method further comprises selecting, or selecting and administering, an FXR agonist or a fibrate, optionally in combination with UDCA.

7. The method of claim 6, wherein the FXR agonist is obeticholic acid.

8. A method for determining the therapeutic effect of a treatment regimen, optionally wherein the treatment regimen comprises ursodeoxycholic acid (UDCA), for a test subject having primary biliary cholangitis, the method comprising:
a) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EpCAM, HAO1, DECR1, HAVCR1 and SCAMP3;
b) repeating step a) using a biological fluid sample obtained from the test subject after treatment for a time interval; and
c) comparing the level of biomarker determined in step a) to that determined in step b), and identifying that the treatment regimen has a therapeutic effect if there is no increase in the level of the one or more biomarker after treatment, or if there is a decrease in the level of the one or more biomarker after treatment.

9. The method of claim 8, comprising determining the level of at least two or three biomarkers, optionally wherein the at least two or three biomarkers are selected from the group consisting of: CCL20, CXCL11, and CXCL10.

10. (canceled)

11. The method of claim 9, wherein the at least two biomarkers comprise CCL20 and CXCL11.

12. The method of claim 8, further comprising determining the level of CXCL9 and/or CCL19 in the biological fluid sample.

13. (canceled)

14. The method of claim 8, wherein the treatment regimen comprises UDCA, and wherein a test subject having no increase in the level of the one or more biomarker after UDCA treatment, or having a decreased level of the one or more biomarker after UDCA treatment, is determined to be a UDCA responder, and a test subject having an increased level of the one or more biomarker after UDCA treatment is determined to be a UDCA non-responder.

15. A method for monitoring the progression of primary biliary cholangitis in a test subject, the method comprising the steps of:
i) determining the level of one or more biomarker in a biological fluid sample from the test subject, wherein the one or more biomarker is selected from the group consisting of: CCL20, CXCL11, CXCL10, CXCL13, IL4RA, IL18R1, CD163, ACE2, CA5A, EpCAM, HAO1, DECR1, HAVCR1 and SCAMP3;
ii) repeating step i) for the same test subject after a time interval; and
iii) comparing the biomarker levels identified in i) with the biomarker levels identified in ii),
wherein a change in the biomarker levels from i) to ii) is indicative of a change in primary biliary cholangitis progression in the test subject.

16. The method of claim 15, comprising determining the level of at least two or three biomarkers, optionally wherein the at least two or three biomarkers are selected from the group consisting of: CCL20, CXCL11, and CXCL10.

17. (canceled)

18. The method of claim 16, wherein the at least two biomarkers comprise CCL20 and CXCL11.

19. The method of claim 15, further comprising determining the level of CXCL9 and/or CCL19 in the biological fluid sample in each of steps i) and ii).

20. (canceled)

21. A method for treating a subject having primary biliary cholangitis, the method comprising: administering an FXR agonist, a fibrate, or ursodeoxycholic acid (UDCA) to a subject, wherein the subject is identified as in need of treatment for primary biliary cholangitis based on having, in a biological fluid sample, an increased level of one or more biomarker selected from the group consisting of: CCL20, CXCL 11, CXCL10, CXCL13, CCL19, IL4RA, IL18R1, CD163, ACE2, CA5A, EpCAM, HAO1, DECR1, HAVCR1 and SCAMP3 compared to a threshold level or range, optionally wherein the FXR agonist is obeticholic acid.

22. The method of claim 21, wherein the subject is undergoing or has previously undergone treatment with ursodeoxycholic acid (UDCA).

23. (canceled)

24. The method of claim 1, wherein the biological fluid sample is a blood sample, optionally wherein the blood sample is a serum or plasma sample.

25. The method of claim 1, wherein the level of biomarker is determined at the protein level, optionally using a process selected from the group consisting of: ELISA assay, immunoblotting, lateral flow assay, protein microarray and mass spectrometry.

26. The method of claim 1, further comprising selecting, selecting and administering, altering, or terminating, a treatment regimen for the test subject based on the comparison of the level of the biomarker with the threshold level or range.

27. The method of claim 26, wherein, for a test subject having an increased level of the one or more biomarker compared to the threshold level or range, the method or use comprises selecting, or selecting and administering, a treatment regimen comprising an FXR agonist or a fibrate, optionally in combination with UDCA.

28. The method of claim 27, wherein the FXR agonist is obeticholic acid.

29. A kit suitable for use in the method of claim 1, the kit comprising:
(i) a detectably labelled agent that specifically binds to CCL20 protein; and
(ii) one or more of:
(a) a detectably labelled agent that specifically binds to CXCL11 protein;
(b) a detectably labelled agent that specifically binds to CXCL10 protein;
(c) a detectably labelled agent that specifically binds to CXCL9 protein; and
(d) a detectably labelled agent that specifically binds to CCL19 protein.

30. The kit of claim 29, further comprising one or more reagents for detecting the detectably labelled agent(s).

31. An assay device suitable for use in the method of claim 1, the device comprising a surface with at least two detectably labelled agents located thereon, optionally wherein the at least two detectably labeled agents are located in separate zones on the surface, wherein the at least two detectably labelled agents are:
(i) a detectably labelled agent that specifically binds to CCL20 protein; and
(ii) one or more of:
(a) a detectably labelled agent that specifically binds to CXCL11 protein;
(b) a detectably labelled agent that specifically binds to CXCL10 protein;
(c) a detectably labelled agent that specifically binds to CXCL9 protein; and
(d) a detectably labelled agent that specifically binds to CCL19 protein.

32. (canceled)
